(19) United States
(12) Patent Application Publication
(10) Pub. No.: US 2024/0202817 A1
(43) Pub. Date: Jun. 20, 2024

(54) CELLULAR PHONE-BASED CONSOLIDATED COMMUNICATION AND FINANCIAL ACCOUNT

(71) Applicant: Y9 Inc., Dubai (AE)

(72) Inventor: Ashish J. Thakkar, Dubai (AE)

(21) Appl. No.: 18/203,907

(22) Filed: May 31, 2023

Related U.S. Application Data
(60) Provisional application No. 63/433,978, filed on Dec. 20, 2022.

(51) Int. Cl. G06Q 40/03; G06Q 20/40; G06Q 40/02; G16H 80/00

(52) U.S. Cl. CPC G06Q 40/03; G06Q 20/40145; G06Q 40/02; G16H 80/00

(57) ABSTRACT

A method for providing access to a multi-platform consolidated communication and financial account. The method includes receiving identifying information from a user, performing a first verification of an identity of the user identity using the identifying information via a first service provider, and a separate second service provider. The method also includes, responsive to a successful first verification and a successful second verification of the identity of the user, creating the multi-platform consolidated communication and financial account. The method further includes providing a communication device to the user, the device providing access to one or more communication networks, creating a record in a user device management system, the record specifying the identity of the user, the account, and a unique identifier of the communication device provided to the user, and linking a payment account of the user to the device to allow for the user's payment via the device.

### BACKGROUND

Traditionally, business that need to assess the risk involved with serving a particular customer, do so in part by verifying financial data for the customer such as monthly income, credit history, bill payment and transactions history, and the like. Analyzing the risk associated with serving the unbanked and underbanked population poses a challenge. Most of these populations do not have credit and transaction history data, which makes it difficult for the banks to evaluate their repayment risk. Many times, such non-traditional prospective borrowers use informal financial practices such as lending among friends and within their own neighborhood. Another common practice is to sell goods in exchange for a promise to pay later when the customer is expected to have money to repay the informal loan. In many of these cases the payment due dates may be uncertain and flexible among the parties and there may be multiple accounts existing for the same person, which also means uncertainty about when these informal lenders will be paid back and from which account. These types of informal, and often unaccountable, transactions involve trust between the parties and may be seen inconvenient or inefficient for merchants that serve the unbanked or underbanked population to monetize their receivables.

### BRIEF DESCRIPTION OF THE DRAWINGS

The accompanying drawings, which are included to provide a further understanding of the disclosed subject matter, are incorporated in and constitute a part of this specification. The drawings also illustrate implementations of the disclosed subject matter and together with the detailed description explain the principles of implementations of the disclosed subject matter. No attempt is made to show structural details in more detail than can be necessary for a fundamental understanding of the disclosed subject matter and various ways in which it can be practiced.

FIG. 1 is an illustrative screen page sequence for customer registration on a consolidated communication and financial account, in accordance with an embodiment of this disclosure.

FIG. 2 is an example interactive logic flow illustrating a method of providing access to device financing via self-onboarding, in accordance with an embodiment of this disclosure.

FIG. 3 is an example interactive logic flow illustrating a method of providing access to device financing via agent assistance, in accordance with an embodiment of this disclosure.

FIG. 4 is an example interactive logic flow illustrating a method of providing access to short term cash loans via the consolidated communication and financial account of FIG. 1, in accordance with an embodiment of this disclosure.

FIG. 5 is an example interactive logic flow illustrating a method of providing access to asset financing via the consolidated communication and financial account of FIG. 1, in accordance with an embodiment of this disclosure.

FIG. 6 is an example interactive logic flow illustrating a method of providing access to buy-now-pay-later (BNPL) financing via the consolidated communication and financial account of FIG. 1, in accordance with an embodiment of this disclosure.

FIG. 7 is an example interactive logic flow illustrating a method of providing access to bill payment financing via the consolidated communication and financial account of FIG. 1, in accordance with an embodiment of this disclosure.

FIG. 8 is an example interactive logic flow illustrating a method of providing access to business loans via the consolidated communication and financial account of FIG. 1, in accordance with an embodiment of this disclosure.

FIG. 9 is an example interactive logic flow illustrating a method of providing access to medium-and-long-term cash loans via the consolidated communication and financial account of FIG. 1, in accordance with an embodiment of this disclosure.

FIG. 10 is an example interactive logic flow illustrating a method of providing access to insurance offerings via the consolidated communication and financial account of FIG. 1, in accordance with an embodiment of this disclosure.

FIG. 11 is an example interactive logic flow illustrating a method of providing access to goal-based savings via the consolidated communication and financial account of FIG. 1, in accordance with an embodiment of this disclosure.

FIG. 12 is an example interactive logic flow illustrating a method of providing access to credit cards via the consolidated communication and financial account of FIG. 1, in accordance with an embodiment of this disclosure.

FIG. 13 is an example interactive logic flow illustrating a method of providing access to debit cards via the consolidated communication and financial account of FIG. 1, in accordance with an embodiment of this disclosure.

FIG. 14 is an example interactive logic flow illustrating a method of providing access to doctors' appointments via the consolidated communication and financial account of FIG. 1, in accordance with an embodiment of this disclosure.

FIG. 15 is an example interactive logic flow illustrating a method of providing access to telehealth via the consolidated communication and financial account of FIG. 1, in accordance with an embodiment of this disclosure.

FIG. 16 is an example interactive logic flow illustrating a method of providing access to pharmacy via the consolidated communication and financial account of FIG. 1, in accordance with an embodiment of this disclosure.

FIG. 17 is an example interactive logic flow illustrating a method of providing access to cash-in financing via the consolidated communication and financial account of FIG. 1, in accordance with an embodiment of this disclosure.

FIG. 18 is an example interactive logic flow illustrating a method of providing access to cash-out-at-agent financing via the consolidated communication and financial account of FIG. 1, in accordance with an embodiment of this disclosure.

FIG. 19 is an example interactive logic flow illustrating a method of providing access to cash-out-at-ATM financing via the consolidated communication and financial account of FIG. 1, in accordance with an embodiment of this disclosure.

FIG. 20 is an example interactive logic flow illustrating a method of providing access to peer-to-peer (P2P) financing via the consolidated communication and financial account of FIG. 1, in accordance with an embodiment of this disclosure.

FIG. 21 is an example interactive logic flow illustrating a method of providing access to transfer service via the consolidated communication and financial account of FIG. 1, in accordance with an embodiment of this disclosure.

FIG. 22 is an example interactive logic flow illustrating a method of providing access to merchant payments via the consolidated communication and financial account of FIG. 1, in accordance with an embodiment of this disclosure.

FIG. 23 is an example interactive logic flow illustrating a method of providing access to bill payments via the consolidated communication and financial account of FIG. 1, in accordance with an embodiment of this disclosure.

FIG. 24 is an example interactive logic flow illustrating a method of providing access to bill payments subscription via the consolidated communication and financial account of FIG. 1, in accordance with an embodiment of this disclosure.

FIG. 25 is an example interactive logic flow illustrating a method of providing access to online payments via the consolidated communication and financial account of FIG. 1, in accordance with an embodiment of this disclosure.

FIG. 26 is an example interactive logic flow illustrating a method of providing access to bulk payment service offerings via the consolidated communication and financial account of FIG. 1, in accordance with an embodiment of this disclosure.

FIG. 27 is an example interactive logic flow illustrating a method of providing access to offers and promotions via the consolidated communication and financial account of FIG. 1, in accordance with an embodiment of this disclosure.

FIG. 28 is an example interactive logic flow illustrating a method of providing access to government payments via the consolidated communication and financial account of FIG. 1, in accordance with an embodiment of this disclosure.

FIG. 29 is an example interactive logic flow illustrating a method of providing access to notifications for delayed payments via the consolidated communication and financial account of FIG. 1, in accordance with an embodiment of this disclosure.

FIG. 30 is an example interactive logic flow illustrating a method of providing access to add bank accounts via the consolidated communication and financial account of FIG. 1, in accordance with an embodiment of this disclosure.

FIG. 31 is an example interactive logic flow illustrating a method of providing access to add credit and debit cards via the consolidated communication and financial account of FIG. 1, in accordance with an embodiment of this disclosure.

FIG. 32 is an example interactive logic flow illustrating a method of providing access to credit scores via the consolidated communication and financial account of FIG. 1, in accordance with an embodiment of this disclosure.

FIG. 33 is an example interactive logic flow illustrating a method of providing access to telecommunication company (telco) top-up via the consolidated communication and financial account of FIG. 1, in accordance with an embodiment of this disclosure.

FIG. 34 is an example interactive logic flow illustrating a method of providing access to gift cards via the consolidated communication and financial account of FIG. 1, in accordance with an embodiment of this disclosure.

FIG. 35 is an example interactive logic flow illustrating a method of providing access to games via the consolidated communication and financial account of FIG. 1, in accordance with an embodiment of this disclosure.

FIG. 36 is an example interactive logic flow illustrating a method of providing access to travel booking via the consolidated communication and financial account of FIG. 1, in accordance with an embodiment of this disclosure.

FIG. 37 is an example interactive logic flow illustrating a method of providing access to pocket money via the consolidated communication and financial account of FIG. 1, in accordance with an embodiment of this disclosure.

FIG. 38 is an example interactive logic flow illustrating a method of providing access to salary advance via the consolidated communication and financial account of FIG. 1, in accordance with an embodiment of this disclosure.

FIG. 39 is an example interactive logic flow illustrating a method of providing access to e-commerce via the consolidated communication and financial account of FIG. 1, in accordance with an embodiment of this disclosure.

FIG. 40 is an example interactive logic flow illustrating a method of providing access to investments accounts via the consolidated communication and financial account of FIG. 1, in accordance with an embodiment of this disclosure.

FIG. 41 is an example interactive logic flow illustrating a method of providing access to chats via the consolidated communication and financial account of FIG. 1, in accordance with an embodiment of this disclosure.

FIG. 42 is an example interactive logic flow illustrating a method of providing access to donations via the consolidated communication and financial account of FIG. 1, in accordance with an embodiment of this disclosure.

### DETAILED DESCRIPTION

Various aspects or features of this disclosure are described with reference to the drawings, wherein like reference numerals are used to refer to like elements throughout. In this specification, numerous details are set forth in order to provide a thorough understanding of this disclosure. It should be understood, however, that certain aspects of disclosure can be practiced without these specific details, or with other methods, components, materials, or the like. In other instances, well-known structures and devices are shown in block diagram form to facilitate describing the subject disclosure.

Various implementations of the disclosed subject matter relate generally to and may provide improvements to apparatus, systems, and methods allowing a customer without a banking account to create and store information related to banking. More particularly, the apparatus, systems and methods allow customers to use a personal mobile device to conduct various transactions and operations, including identity management, account creation and management, banking and other financial transactions, and the like. Embodiments disclosed herein may operate without the need to use physical cash checking locations for the transactions, for example by delegating to each consumer control over the cash checking process, thus eliminating the need for a physical store or other location, in-person employees, safety boxes, security systems, and the like. In some embodiments, consumers can self-enroll using the mobile application or register at a number of authorized physical enrollment locations, usually independent, local establishments such as mobile phone dealers, convenience stores, and insurance brokers. The result is an improved solution for unbanked and under-banked consumers to manage their finances by providing cheaper cost of service, higher ability to transact anywhere and anytime, more secure ways to store value other than cash, and leveraging new sources of information to extend credit.

People in many places of the world do not have banking accounts. These people generally conduct their financial transactions in cash. Without a bank account, it may be difficult to pay for some purchases such as online internet products. It may also be difficult to transfer money to a relative or another person at a distant location. There remains a need for better methods and systems for executing financial transactions.

The present disclosure provides a way to have funds transferred to a personal mobile customer device such as a cellphone to allow the users to use their cellphones to perform many functions traditionally performed with banking accounts. For example, the users may make payments at stores using their phone, may make payments over the internet and perform other traditional banking transactions using their phones. In some embodiments, the phone device may operate like to a prepaid credit or debit card with similar credentials to the prepaid debit or credit card stored in the phone and associated with a unique and singular account on a trusted bank server. Thus, embodiments of the present disclosure provide methods of loading credit into a personal electronic device or phone, without the need to open a formal bank account, is an effective solution for unbanked customers.

In an aspect of the disclosed subject matter, a method for providing access to a multi-platform consolidated communication and financial account is disclosed. The method may include receiving identifying information from a user, performing a first verification of the user identity such as a “know your customer” (KYC) verification, using the identifying information via a first service provider, and a second service provider separate from the first service provider. The method may also include, responsive to a successful first verification and a successful second verification of the identity of the user, creating a multi-platform consolidated communication and financial account. The method may further include providing a communication device to the user, the communication device providing access to one or more communication networks, creating a record in a user device management system, the record specifying the identity of the user, the account, and a unique identifier of the communication device provided to the user, and linking a payment account of the user to the device to allow for payment by the user via the device.

The method may include preventing opening a second account by the same user and responsive to an attempt by the user to create the second account, generating an error.

The method may include prior to performing the first verification of the user, receiving a request from the user to identify an authorized agent to establish an account for the user on the device. The step of performing the first verification of the user may further include receiving confirmation of the identifying information from an agent in physical proximity to the user. The confirmation indicates that the agent has independently verified the accuracy of the identifying information. The step of performing the first verification of the user may further include sending the identifying information to a telephone service provider, receiving, from the telephone service provider, a confirmation that the identifying information matches a user record of an account at the telephone service provider.

The method may include receiving a request for a deferred payment plan from the device for purchase of an item from a merchant, responsive to the request, confirming an eligibility of the user, responsive to confirming that the user is eligible, verifying a biometric signature of the user via a third service provider separate from the first service provider and the second service provider. The method may further include responsive to successful verification of the biometric signature of the user, providing payment for the item to the merchant and adding the deferred payment plan to an account of the user associated with the device.

The method may include providing access to device financing via self-onboarding on the multi-platform consolidated communication and financial account. The step of providing access to device financing may include the user signing up on the user device management system, the user providing identifying information to the user device management system, appraising a credit eligibility of the user via a third service provider separate from the first service provider and the second service provider, responsive to user being eligible for credit, forwarding the user to a registering agent, the user completing onboarding with assistance from the registering agent. The method may include the user paying a dollar amount from a user account as cost of a membership via the user device management system, and updating the user account.

The method may include providing access to device financing via an agent-assisted onboarding on the multi-platform consolidated communication and financial account. The step of providing access to device financing may include the user signing up on the user device management system, the user providing identifying information to the user device management system, a registering agent receiving the identifying information, the registering agent validating the user's identity, the registering agent appraising a credit eligibility of the user via a third service provider separate from the first service provider and the second service provider, and responsive to the user being eligible for credit, completing onboarding of the user. The method may further include the user paying a dollar amount from a user account as cost of a membership with consumer based financial application, and updating the user account.

The method may include providing access to a loan via the multi-platform consolidated communication and financial account. The loan may include at least one of a short-term cash loan, an asset financing loan, a BNPL loan, a bill payment financing, a business loan, and a medium-term and long-term cash loan. The step of providing access to a loan may include displaying a plurality of financial offerings available to the user, the user selecting a loan option from the plurality of financial offerings, appraising a credit eligibility of the user via a third service provider separate from the first service provider and the second service provider, responsive to user being eligible for credit, forwarding the user to a loan agent. The method may further include the user completing a loan registration with assistance from the loan agent, the user paying a dollar amount from a user account as cost of the loan, updating the user account, monitoring for timely payments to the loan, and automatically offering additional loan based on the payments to the loan. The appraising a credit eligibility of the user may include obtaining a credit score of the user, the credit score of the user being above a minimum threshold, initiating the loan to the user's authenticated identity.

The method may include, responsive to determining that the loan is in default, blocking access to the one or more communication networks by the device.

The method may include, responsive to successful use of the device and receipt of a minimum number of timely loan payments, offering a second loan to the user, wherein terms of the second loan are based on a user history of usage of the device.

The method may include providing access to insurance via the multi-platform consolidated communication and financial account. The step of providing access to insurance may include displaying a plurality of financial offerings available to the user, the user selecting an insurance option from the plurality of financial offerings, appraising an insurance eligibility of the user via a third service provider separate from the first service provider and the second service provider, responsive to user being eligible for insurance, forwarding the user to an insurance agent, the user completing an insurance registration with assistance from the insurance agent. The method may further include the user paying a dollar amount from a user account as cost of the insurance, updating the user account, monitoring for timely payments to the insurance, and automatically offering additional insurance based on the payments to the insurance.

The method may include providing access to goal-based savings via the multi-platform consolidated communication and financial account. The step of providing access to goal-based savings may include displaying a plurality of financial offerings available to the user, the user selecting a goal-based savings option from the plurality of financial offerings, appraising a credit eligibility of the user via a third service provider separate from the first service provider and the second service provider, responsive to user being eligible for credit, forwarding the user to a goal-based savings registering agent, the user completing a goal-based saving scheme registration with assistance from the goal-based savings registering agent. The method may further include the user paying a dollar amount from a user account as cost of the goal-based saving scheme, updating the user account, monitoring for timely payments to the goal-based saving scheme, and automatically offering additional goals based on the payments to the goal-based saving scheme.

The method may include providing access to a consumer card via the multi-platform consolidated communication and financial account. The consumer card may include a credit card or a debit card. The step of providing access to a consumer card may include displaying a plurality of financial offerings available to the user, the user selecting a consumer card option from the plurality of financial offerings, appraising a credit eligibility of the user via a third service provider separate from the first service provider and the second service provider, responsive to user being eligible for credit, forwarding the user to a consumer card registering agent, and the user completing consumer card registration with assistance from the consumer card registering agent. The method may also include the user paying a dollar amount from a user account as cost of the consumer card service, updating the user account, monitoring for timely payments to the consumer card, and automatically offering additional loans based on the payments to the consumer card.

The method may include providing access to a telehealth service via the multi-platform consolidated communication and financial account. The step of providing access to a telehealth service may include displaying a plurality of healthcare offerings available to the user, the user selecting a telehealth option from the plurality of healthcare offerings and selecting a doctor, checking an availability of the telehealth service using a telehealth doctor's calendar, the telehealth service offered by a third service provider separate from the first service provider and the second service provider, reserving an appointment on the telehealth doctor's calendar and confirming the appointment with the user, scheduling the appointment on the doctor's calendar and on the user's calendar. The method may also include the user paying a dollar amount from a user account as cost of the telehealth service, and updating the user account.

The method may include providing access to a doctor's appointments via the multi-platform consolidated communication and financial account. The step of providing access to a doctor's appointments may include displaying a plurality of healthcare offerings available to the user, the user selecting a doctor's appointments option from the plurality of healthcare offerings and selecting a doctor, checking an availability of doctor's from the doctor's calendar via a third service provider separate from the first service provider and the second service provider, reserving an appointment on the doctor's calendar and confirming the appointment with the user, scheduling the appointment on the doctor's calendar and on the user's calendar. The method may also include the user paying a dollar amount from a user account as cost of the telehealth service, and updating the user account.

The method may include providing access to a pharmacy via the multi-platform consolidated communication and financial account. The step of providing access to a pharmacy may include displaying a plurality of healthcare offerings available to the user, the user selecting a pharmacy from the plurality of healthcare offerings, the pharmacy operated by a third service provider separate from the first service provider and the second service provider, the user selecting a product from the pharmacy for purchase The method may also include the user paying a dollar amount from a user account as price of the product, updating the user account, and shipping the product to the user.

The method may include providing access to a merchant payment via the multi-platform consolidated communication and financial account. The step of providing access to a merchant payment may include displaying a plurality of options to the user to make the merchant payment, displaying instructions on how to make the merchant payment via a third service provider separate from the first service provider and the second service provider. The method may also include the user scanning a QR code related to the merchant payment, the user paying the dollar amount from a user account to make the merchant payment, and updating the user account.

The method may include providing access to a bill payment process via the multi-platform consolidated communication and financial account. The step of providing access to a bill payment process may include displaying a plurality of options to the user to make the bill payment, displaying instructions on how to make the bill payment via a third service provider separate from the first service provider and the second service provider. The method may also include the user selecting one of the plurality of options to make the bill payment, the user entering a bill payment account number and a dollar amount to pay, the user paying the dollar amount from a user account, and updating the user account.

The method may include providing access to government payments via the multi-platform consolidated communication and financial account. The step of providing access to government payments may include displaying a plurality of options to the user to make government payments, displaying instructions on how to make government payments via a third service provider separate from the first service provider and the second service provider, the user selecting one of the plurality of options to make the government payment. The method may also include the user entering a user account number to make the payment from and a dollar amount to pay, the user paying the dollar amount from the user account, updating the user account, and the user optionally subscribing for an automated payment on a recurring due date.

The method may include providing access to add a bank account via the multi-platform consolidated communication and financial account. The step of providing access to add a bank account may include displaying a plurality of options to the user to add a bank account, displaying instructions on how to add the bank account via a third service provider separate from the first service provider and the second service provider. The method may also include the user selecting one of the plurality of options to add the bank account, the user entering an associated number of the bank account, and linking the bank account with a purchase account of the user, and making the bank account available for future transactions of the user.

The method may include adding a consumer card via the multi-platform consolidated communication and financial account. The consumer card may be a credit card or a debit card. The step of adding a consumer card may include displaying a plurality of options to the user to add a consumer card, displaying instructions on how to add a consumer card via a third service provider separate from the first service provider and the second service provider. The method may also include the user selecting one of the plurality of options to add the consumer card, the user entering an associated number of the consumer card, and linking the consumer card with a purchase account of the user, and making the consumer card available for future transactions of the user.

The method may include providing access to credit score via the multi-platform consolidated communication and financial account. The step of providing access to credit score may include displaying a plurality of options to the user to check a credit score of the user via a third service provider separate from the first service provider and the second service provider. The method may also include the user selecting one of the plurality of options to check the credit score, sending a credit score check request to a credit score system, receiving the credit score of the user from the credit score system, and displaying the credit score of the user.

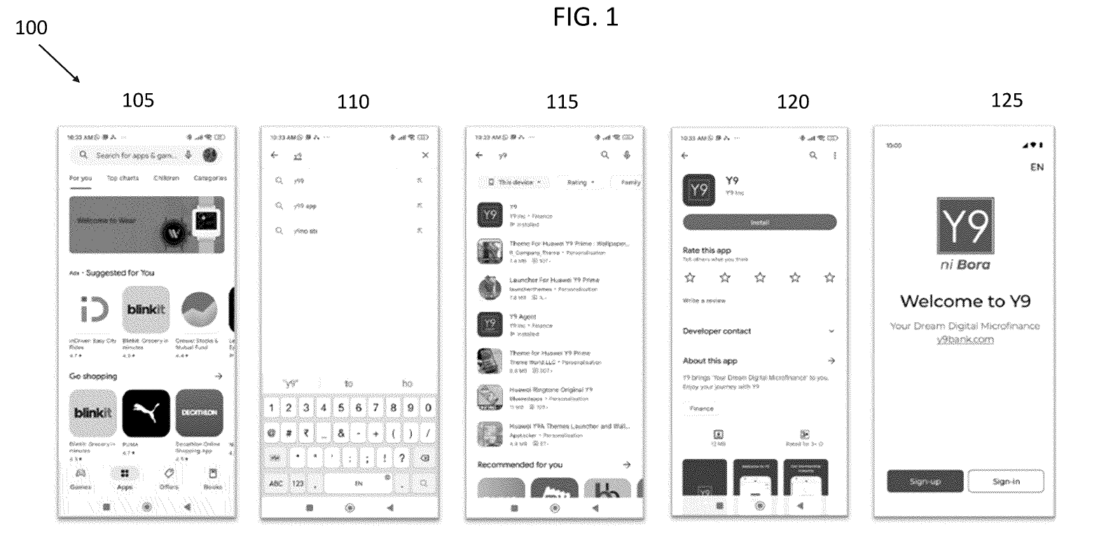

FIG. 1 is an illustrative screen page sequence 100 for customer registration on a consolidated communication and financial account (also referred to as “consumer app”), in accordance with an embodiment of this disclosure. A first example screen 105 is a representative view of an initial starting point of use by a customer of the consumer app. A number of different applications (also referred to as “app”) are displayed as available for the customer's access, as is common in any digital app store. A second example screen 110 illustrates how the customer can search for the consumer app, represented as “Y9”, of this disclosure in the search bar of a common web browser. A third example screen 115 illustrates a number of search hits including the Y9) Consumer app available for installation and access by the customer. A fourth example screen 120 illustrates an example installation page of the Y9 Consumer app. A fifth example screen 125 illustrates an example “Sign-up” or “Sign-in” page after the customer installs the Y9 app. A similar process may be used to install an “agent” version of the app for use by agents of the system, for example to perform local validation of user identity and other operations.

The home page of the customer may display several finance related utilities, such as money wallets that may be a source for loan fund into the wallet (bank account, debit card, credit card), transfer funds from one wallet to another, online transaction for utility billing, mobile recharge and the like.

The home page of the customer may display several referral options and the customer may send the referral codes to any of the customer's contacts. An assisting agent may use the referral codes during the onboarding process. Further, the customer may access the referral benefit in the form of credibility points (CP) or similar credit towards to settle the customer's due amount. The credibility points may be earned by making timely payments and there may be several incentives based on the credibility points earned. For example, if a customer earns 100) CPs, the customer may qualify for a subsequent loan. The consolidated communication and financial account may preload a “pera-wallet” which may be, for example, a blockchain wallet, which may include features such as a blockchain “play2earn” gaming platform or other features in which users can complete tasks, use digital products or services, provide review information, or the like to obtain digital currency, digital goods, or the like. For example, customers may play games and earn money that is placed into their pera-wallet, and which then can be seamlessly transferred to their wallets.

The consolidated communication and financial account may also onboard a chain of merchants to enable consolidated account access and management and electronic payment, such as via NFC or QR code. The consolidated communication and financial account typically also may combine utility provider accounts, for example to facilitate payment of routine monthly bills and government payments such as taxes. The consolidated communication and financial account may also provide financing options for monthly bills and offer daily payment options to the customers. The consolidated communication and financial account also may provide insurance services to customer and instead of them paying for a year in advance daily, weekly, or monthly payments may be provided.

The consolidated communication and financial account may issue virtual and physical credit and debit cards. The consolidated communication and financial account may provide saving schemes linked to tangible outcomes, such as education savings. In an example scheme, a customer may save a certain amount and instead of receiving an annual interest, the consolidated communication and financial account may provide tangible benefits for the customers such as school uniforms, shoes and books to their children certain times a year. The consolidated communication and financial account may have a locked-in “Loyalty and Rewards” program which may include credibility points and several other benefits and incentives.

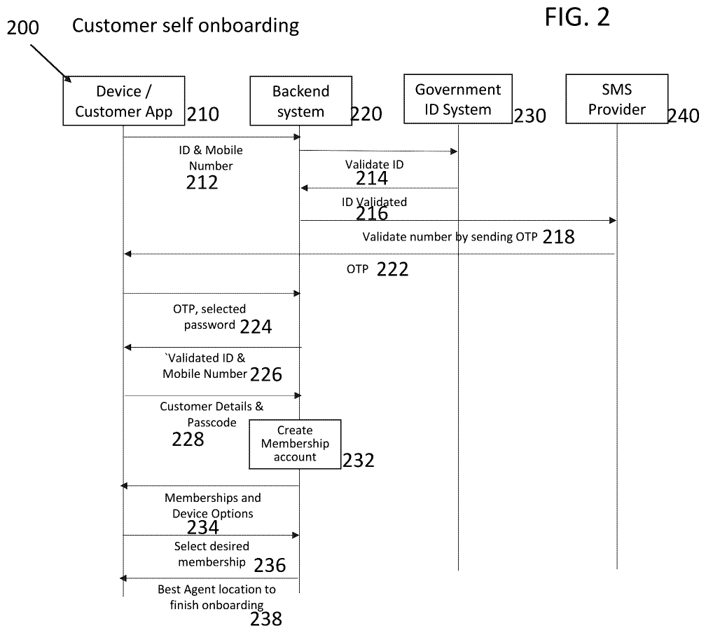

FIG. 2 is an example interactive logic flow 200 illustrating a method of providing access to device financing via self-onboarding on the consolidated communication and financial account of FIG. 1, in accordance with an embodiment of this disclosure. The example interactive logic flow 200 shows communications and data transfer between a number of interaction stations, such as a customer application 210, a back-end system 220, a Government ID system 230, an SMS provider 240 and the like. It will be understood that, in different system architectures, equivalent entities for those shown in FIG. 2 and other logic flows provided herein may be used. For example, the SMS provider 240 may be replaced with an equivalent messaging service or system provider. As another example, in some jurisdictions the Government ID system 230 may be a system of a particular branch of local, state, county, provincial, or federal government or it may be a private business sub-contracted to and/or managed by a government agency, or the like. The “Customer Self-Onboarding” process begins when a new customer clicks on a “Sign Up” button, as in 212. The customer is requested to enter identifying information such as a “customer national ID” number, a mobile number and the details of a related mobile service provider. The customer clicks on a checkbox and consents that the consolidated communication and financial account may check with the respective authorities for the authenticity of the identifying information.

A three-way “know-your customer” (KYC) or equivalent validation of the customer may be performed to verify the identity of the customer. First, the customer ID may be validated. A back-end system of the consolidated communication and financial account (also referred to as “the back-end system”) is connected to a government identification system. The customer national ID and the customer's fingerprints may be sent to the government identification system for validation, as in 214. The government identification system matches the ID and the fingerprints and validates the identity of the customer, as in 216.

Second, the mobile number of the customer may be validated. The back-end system originates and sends the customer a Short Message/Messaging Service (SMS) with a one-time passcode (OTP) to verify the mobile number provided by the customer, as in 218. The SMS and the OTP received by the customer are entered into the application and validated against the generated OTP, as in 222, 224, and 226.

Third, the details of the customer may be validated with the customer's mobile service provider, as in 228. Specifically, the name of the customer and the mobile number may be sent to the mobile service provider for validation. The mobile service provider may check the name provided against the registered mobile number and verify that the customer identifier matches a customer record of an account maintained in the mobile service provider's database. Customer subsequently enters additional personal details such as, date of birth, gender, profession, email ID, address, organization, region, district, country and the like. In one embodiment, the name of the country may be automatically populated based on the location provided by the customer.

Credit scores of the customer may be checked next, to appraise the financial eligibility of the customer. There may be several parameters that are checked to ascertain the financial eligibility. First, the back-end system may check the customer ID, the name and the mobile number to check for any customer blacklisting. Second, the back-end system may check the customer ID, name and mobile number for any customer delinquency. Third, an analytical algorithm for credit scoring may determine the credit score for the customer using data provided by the mobile service provider (such as, data usage, top up, calls, SMS, active rate, time since activation etc.), mobile money wallet data (such as, balance, transfers, loans, top ups, usage, active rate etc.), financial data (such as, accounts, loans, repayments, payments delays etc.), government credit check information using customer government financial data (such as, loans, credit cards, payments etc.) and the like.

KYC and credit checks as disclosed herein may be performed in compliance with any applicable data privacy rules based on the geographic location of the service provider and/or the end user customer. At the end of the credit check, if the customer is not found eligible, the consumer financial application may send a message to the customer that the customer may reach out again to the financial institution after a few months. The same message may also be sent by an SMS. On the other hand, after a successful KYC verification if the credit score of the customer is found to be above a minimum threshold, a membership may be created for the customer, as in 232. Several device and membership options may be shared with the customer, as in 234. Further, if a user attempts to opening a second account, a logic check is performed by the systems on the identification details and an error is generated. In some embodiments, it may be preferred for each human user to be prevented from creating more than one account. That is, each account in a system as disclosed herein may be connected to a single human user and, similarly, each human user may be connected to at most one account on the system.

At this point, the customer may select a particular device and a membership option. In some embodiments, verification by a local agent may be desirable or required to complete creation of the user's account and enrollment in the system. In that case, the customer is shown a “agents nearby” option. Specifically, the device may display a list of agents, the contact details of the agents, and the distance between the customer's location and the agents' locations. Once the customer selects an agent, as in 236, navigation directions to the agent may be displayed using the location capabilities installed on the device and standard “map” applications. The customer subsequently reaches out to an agent and completes the onboarding process, as in 238.

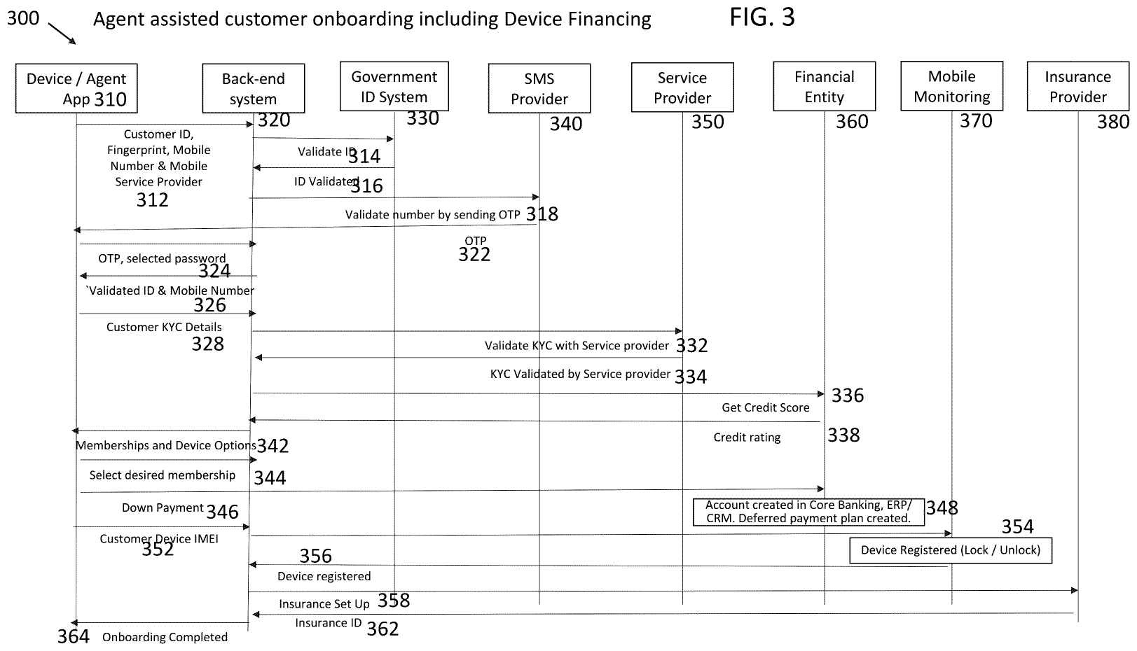

FIG. 3 is an example interactive logic flow 300 illustrating a method of providing access to device financing via assistance of an agent, in accordance with an embodiment of this disclosure. Referring to FIG. 3, the interactive logic flow 300 includes a number of interaction stations, such as a customer application 310, a back-end system 320, a Government ID system 330, an SMS provider 340, a KYC verification service provider 350, a financial entity 360, a mobile monitoring agency 370, an insurance provider 380 and the like. The “Agent Assisted Customer Onboarding including Device Financing” process begins when an agent receives identifying information from a customer, as in 312. The identifying information may include the a “customer national ID” number, a mobile number and the related mobile service provider. The agent explains the KYC and credit check process to the customer and receives a consent from the customer to check KYC and credit by the customer's clicks on a checkbox.

A four-way KYC validation of the customer may be performed using the identifying information to verify the identity of the customer. First, the agent, in physical proximity to the customer, receives the identifying information from the customer, as in 312. The agent verifies the information, as in 314 and submits confirmation of the identifying information, as in 316. The confirmation indicates that the agent has independently verified the accuracy of the identifying information. Second, the customer ID may be validated as explained above in relation to the method of FIG. 2. Third, the mobile number of the customer may be validated, as in 318. 322, 324, and 326 and as explained above in relation to the method of FIG. 2. Fourth, the details of the customer may be validated with the KYC verification service provider, as in 328, 332, and 334 as explained above in relation to the method of FIG. 2.

The agent may then proceed to enter additional personal details of the customer and check the financial eligibility of the customer as explained above in relation to the method of FIG. 2.

KYC and credit checks may be performed in compliance with applicable data privacy rules at 336, as previously disclosed in relation to the self-onboarding process. At the end of the credit check, if the customer is not found eligible, the consumer financial application shares the message that the customer may reach out again to the financial institution after a few months. The same message may also be sent by an SMS or other electronic messaging system. On the other hand, after a successful KYC verification if the credit score of the customer is found to be above a minimum threshold, as in 338, a membership may be created for the customer, as in 342. Several device and membership options may be shared with the customer, as in 344. At this point, the customer may select a particular device and a membership option.

In some embodiments, the electronic device provided to the end user customer may be provided free of charge; in others it may be provided immediately but payment may be required from the user. In some cases, the need for a down-payment amount may be communicated to the customer. For example, an SMS message may be sent to the to provide a link and instructions to pay the down-payment amount using the customer's bank or mobile money account. In one embodiment, a default payment account of the customer may be linked to the device to allow for payment of the down-payment amount via the consolidated communication and financial account.

Subsequently, the back-end system may check for confirmation of the down-payment, as in 346. Once the down-payment is confirmed, a loan account may be created in the core banking system, and/or an enterprise resource planning (ERP) system and/or a customer relationship management (CRM) system, as in 348. A deferred payment plan may be created. The assisting agent may scan a unique identifier such as the international mobile equipment identity (IMEI) of the selected device, as in 352. The device IMEI may be sent to the back-end system, as in 354, creating a record in a customer device management system (also known as “Mobile Monitoring System”). The record specifies the identity of the customer, the loan amount, and the IMEI. The device may be unlocked and prepared for the customer's use, as in 356.

In some embodiments, device information may be sent to an insurance provider and an insurance process may be implemented for the device provided to the customer. Usually an insurance ID may be provided by the insurance company, as in 358 and a corresponding record may be created in the customer database, as in 362.

The agent may hand over the device to the customer and the customer may power the device on and connect to the internet. The customer may get the device configured to communicate and transact on several financial institutions of the customer, such as a bank account, credit scoring engine and the like. The agent may explain the benefits of the free e-books offered by the consolidated communication and financial account and the customer may sign in by setting a new passcode.

Once the device has been provided to the end user customer and any optional features have been enabled and/or any optional processes (such as payment, insurance, and the like) have been completed, he onboarding process is complete at this point, as in 364.

In embodiments where the device is provided to the user immediately by way of a loan to the customer for the cost of the device, the customer may link a payment account to the device to allow for payment of the loan. Customers typically may have several options, such as “pay due amount”, “pay daily payment amount”, “pay total loan amount”, and “pay custom amount” available to make the payment. Next, the customer may select one or more from a list of mobile money wallets that may include “choice of payment wallet 1”, “choice of payment wallet 2”, and “choice of payment wallet 3”. All transactions completed by the customer may get updated into loan account of the customer.

The consolidated communication and financial account continuously checks for timely payments and responsive to determining that any loan is in default, the customer's access to the device may be blocked. Further, responsive to successful use of the device and receipt of a minimum number of timely loan payments, the system may offer a subsequent (second or third and so forth) loan to the customer. The terms of the subsequent loans may be decided based on a history of the customer's usage of the device.

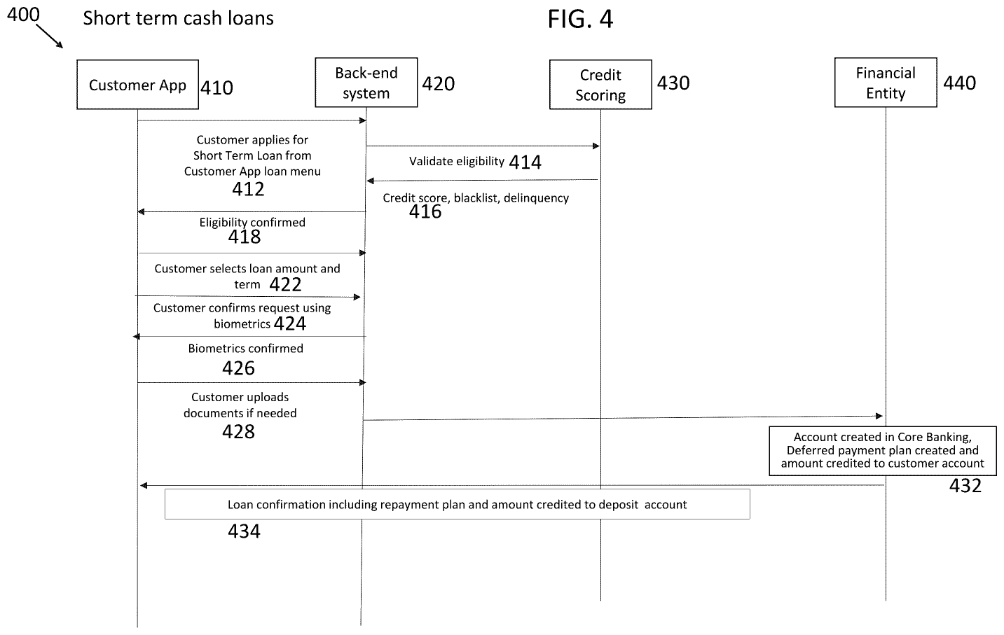

FIG. 4 is an example interactive logic flow 400 illustrating a method of providing access to short term cash loans via the consolidated communication and financial account of FIG. 1, in accordance with an embodiment of this disclosure. Referring to FIG. 4, the interactive logic flow 400 includes a number of interaction stations, such as a customer application 410, a back-end system 420, a credit scoring agency 430, a financial entity 440 and the like. The “Short Term Cash Loans” process begins when an option to apply for loans is displayed to the customer on the home screen. Once clicked, all loans available to the customer are displayed on the screen, as in 412. One of the options on the loan menu may be “Short Term Cash Loans”. Once the customer selects this option, as in 414, credit scores of the customer may be checked next to appraise the eligibility of the customer, as in 416 and as explained above in relation to the method of FIG. 2. After eligibility is confirmed (credit score of the customer being above a minimum threshold), as in 418, the short-term cash loans options are shared with the customer.

At this point, the customer may select a loan amount and a term of the loan, as in 422. The customer may confirm a request using instant biometrics, as in 424. The instant biometrics may be compared with the ones submitted by the customer during onboarding, face or fingerprint depending on device and selection. Once the biometrics are approved, as in 426, there may be a request for additional documents, as in 428. Subsequently a loan account may be created in the core banking system, and/or an ERP system and/or a CRM system, as in 432. A deferred payment plan may be created. Further, the customer may be able to view a summary of the loan application that includes the requested loan amount and the approved limit. Once consented, the loan amount may be credited to the customer's account, as in 434. At this point, the customer may link a payment account and make necessary payments as explained above in relation to the method of FIG. 3.

As previously indicated, embodiments disclosed herein allow for a single account, tied to a specific electronic device, to be used by the end user customer to perform various transactions. The operation and result of those transactions may further be used to adjust the user's profile in the system, for example to change the user's credit rating, payment history, available devices and communication services (such as cellular accounts and the like), and to update associated attributes such as the interest rate or financing charges applied to the user's accounts, payment and service level options, and the like. For example, if an end user customer applies for and receives a loan or on-the-spot financing, the amount and interest rate given to the user may be determined, at least in part, based on the user's previous behavior in the system. This may operate in a similar manner to conventional credit scores, i.e., net “positive” behavior (that would increase a conventional credit score) may result in higher amounts, more complex financial products, and/or lower interest rates being offered to the user, while net “negative” behavior (that would decrease a conventional credit score) may result in the opposite. Other behavior may be included in such determinations, such as whether or how often a user has reported the associated personal electronic device lost or damaged, the number of accounts connected within the communication and financial account, the type of such accounts, the user's average weekly or monthly expenditures, and the like. More generally, any activity performed through the app as disclosed herein may be accounted and weighted when determining which products or services to offer to a particular user according to embodiments disclosed herein.

Various examples of the types of services that may be made available to users are described below, but they are not intended to be limiting on the type, nature, or operation of services that may be made available to users in embodiments disclosed herein.

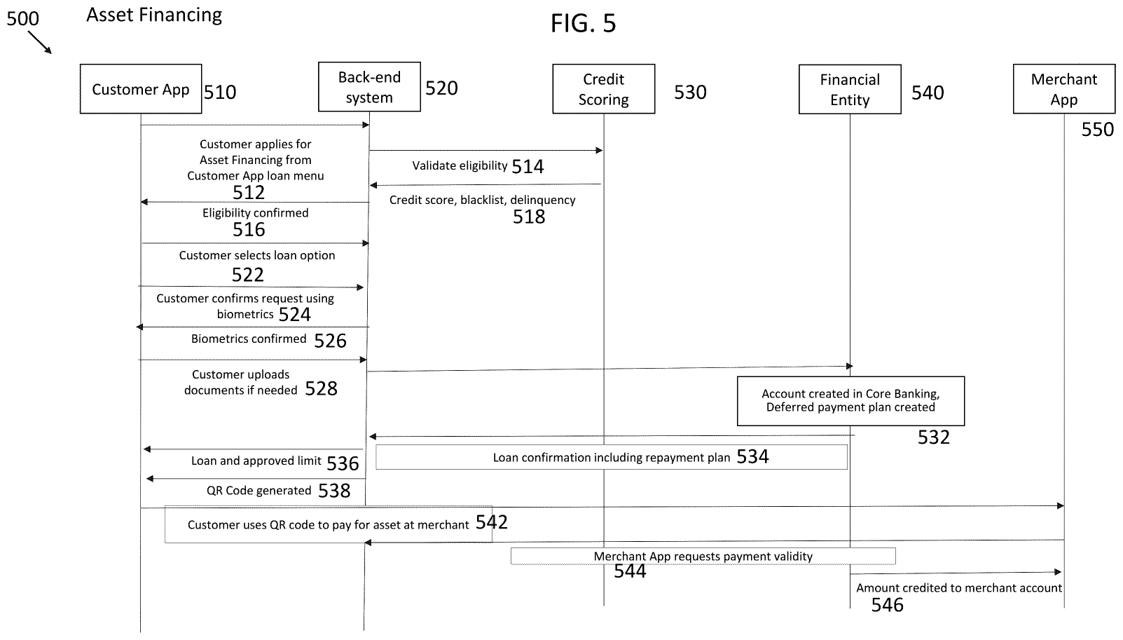

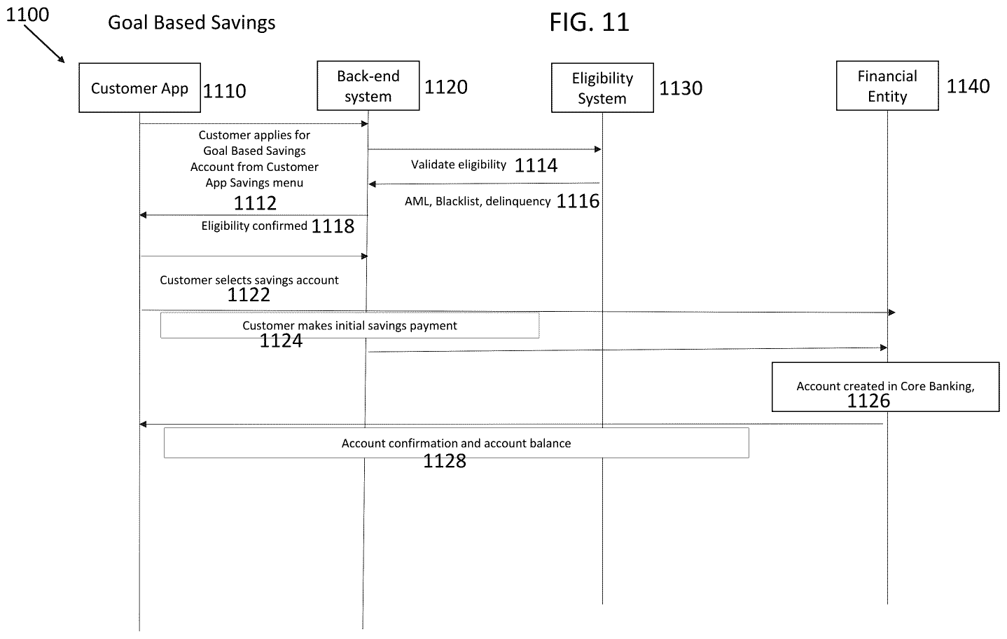

FIG. 5 is an example interactive logic flow 500 illustrating a method of providing access to asset financing via the consolidated communication and financial account of FIG. 11, in accordance with an embodiment of this disclosure. The interactive logic flow 500 includes a number of interaction stations, such as a customer application 510, a back-end system 520, a credit scoring agency 730, a financial entity 740, a merchant application and the like. The “Asset Financing” process begins when an option to apply for loans displayed to the customer on the home screen. Once clicked, all loans available to the customer are displayed on the screen, as in 512. One of the options on the loan menu may be “Asset Financing”. Once the customer selects this option, as in 514, credit scores of the customer may be checked next to appraise the eligibility of the customer, as in 516 and as explained above in relation to the method of FIG. 2. After eligibility is confirmed (credit score of the customer being above a minimum threshold), as in 518, the insurance options are shared with the customer.

At this point, the customer may select a loan option for a particular asset of interest, such as a television, a motorbike and the like, as in 522. The customer may confirm a request using instant biometrics, as in 524. The instant biometrics may be compared with the ones submitted by the customer during onboarding, face or fingerprint depending on device and selection. Once the biometrics are approved, as in 526, there may be a request for additional documents, as in 528. Subsequently a loan account may be created in the core banking system, and/or ERP system and/or a CRM system, as in 532. A deferred payment plan may be created, as in 534. Further, the customer may be able to view a summary of the loan application that includes the requested loan amount and the approved limit, as in 536.

Subsequently, a QR code with an approved amount may be generated and made available in the application to be used for purchase of the asset from a merchant, as in 538. The Customer may also have an option to view or download a list of participating stores and the steps to use the approved amount. The customer may see the QR code in a “My loans” section or under an “Asset Financing” tab on the home page. The customer may reach out to a merchant for purchase of a particular asset and inform the QR code the merchant at the time of payment. The merchant may open a “Merchant app” and select received payment and scan the QR code provided by the customer, as in 542. The Merchant App may request the back-end system for validation of the payment, as in 544. Once validated, the amount may be credited to the merchant's account, as in 546. At this point, the customer may link a payment account and make necessary payments as explained above in relation to the method of FIG. 3.

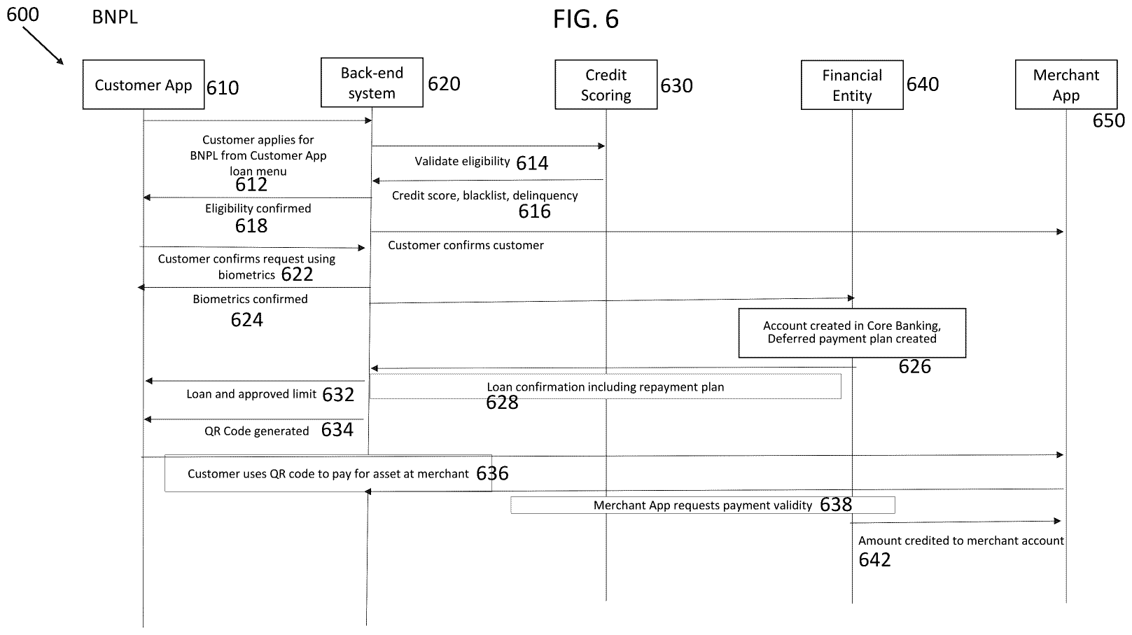

FIG. 6 is an example interactive logic flow 600 illustrating a method of providing access to buy-now-pay-later (BNPL) financing via the consolidated communication and financial account of FIG. 1, in accordance with an embodiment of this disclosure. Referring to FIG. 6, the interactive logic flow 600 includes a number of interaction stations, such as a customer application 610, a back-end system 620, a credit scoring agency 630, a financial entity 640), a merchant application 650 and the like. The “Buy Now Pay Later” process begins when an option to apply for loans is displayed to the customer on the home screen. Once clicked, all loans available to the customer are displayed on the screen, as in 612. One of the options on the loan menu may be “Buy-now-pay-later (BNPL)”. Once the customer selects this option, as in 614, credit scores of the customer may be checked next to appraise the eligibility of the customer, as in 616 and as explained above in relation to the method of FIG. 2. After eligibility is confirmed (credit score of the customer being above a minimum threshold), as in 618, the insurance options are shared with the customer.

At this point, the customer may scan a QR code from a merchant and enter an amount as in 622. The customer may confirm a request using instant biometrics, as in 624. The instant biometrics may be compared with the ones submitted by the customer during onboarding, face or fingerprint depending on device and selection. Once the biometrics are approved, as in 626, there may be a request for additional documents, as in 628. Subsequently a loan account may be created in the core banking system, and/or ERP system and/or a CRM system, as in 632. A deferred payment plan may be created, as in 634. Further, the customer may be able to view a summary of the loan application that includes the requested loan amount and the approved limit, as in 636. Once consented, the loan amount may be credited to the customer's account.

Subsequently, a QR code with an approved amount may be generated and made available in the application to be used for purchase of the asset from a merchant, as in 638. The merchant may open a “Merchant app” and select received payment and scan the QR code provided by the customer. The Merchant App may request the back-end system for validation of the payment, as in 642. Once validated, the amount may be credited to the merchant's account, as in 644. At this point, the customer may link a payment account and make necessary payments as explained above in relation to the method of FIG. 3.

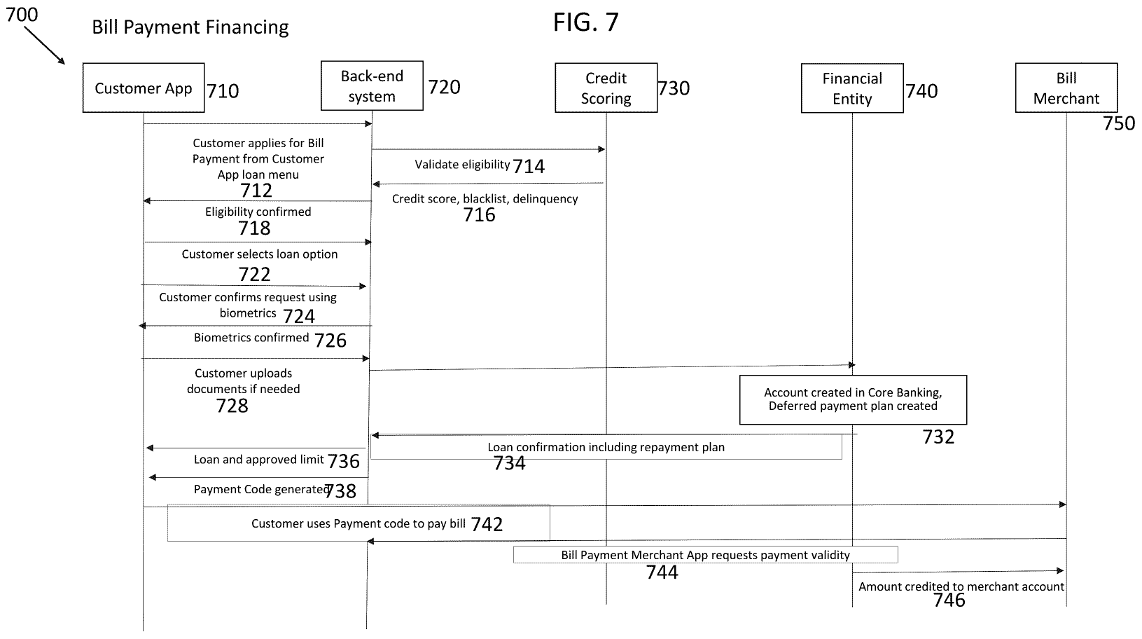

FIG. 7 is an example interactive logic flow 700 illustrating a method of providing access to bill payment financing via the consolidated communication and financial account of FIG. 1, in accordance with an embodiment of this disclosure. Referring to FIG. 7, the interactive logic flow 700 includes a number of interaction stations, such as a customer application 710, a back-end system 720, a credit scoring agency 730, a financial entity 740, a bill merchant 750) and the like. The “Bill Payments Financing” process begins when an option to apply for loans is displayed to the customer on the home screen. Once clicked, all loans available to the customer are displayed on the screen, as in 712. One of the options on the loan menu may be “Bill Payment Financing”. Once the customer selects this option, as in 714, credit scores of the customer may be checked next to appraise the eligibility of the customer, as in 716 and as explained above in relation to the method of FIG. 2. After eligibility is confirmed (credit score of the customer being above a minimum threshold), as in 718, the insurance options are shared with the customer.

At this point, the customer may select a loan option, as in 722 and confirm the request using instant biometrics, as in 724. The instant biometrics may be compared with the ones submitted by the customer during onboarding, face or fingerprint depending on device and selection. Once the biometrics are approved, as in 726, there may be a request for additional documents, as in 728. Subsequently a loan account may be created in the core banking system, and/or ERP system and/or a CRM system, as in 732. A deferred payment plan may be created, as in 734. Further, the customer may be able to view a summary of the loan application that includes the requested loan amount and the approved limit, as in 736. Once consented, the loan amount may be credited to the customer's account.

Subsequently, a payment code with the amount may be generated and made available in the application to be used for payment of a merchant's bills, as in 738. The customer may see the payment code in “My loans” section or under “Bill Payment” on home page. The customer may select a bill payee merchant and select an option to pay via the payment code. The customer may enter the bill payment code in bill payee merchant section, as in 742. The bill payee merchant may request the back-end system for validation of the payment, as in 744. Once validated, the amount may be credited to the merchant's account, as in 746. At this point, the customer may link a payment account and make necessary payments as explained above in relation to the method of FIG. 3.

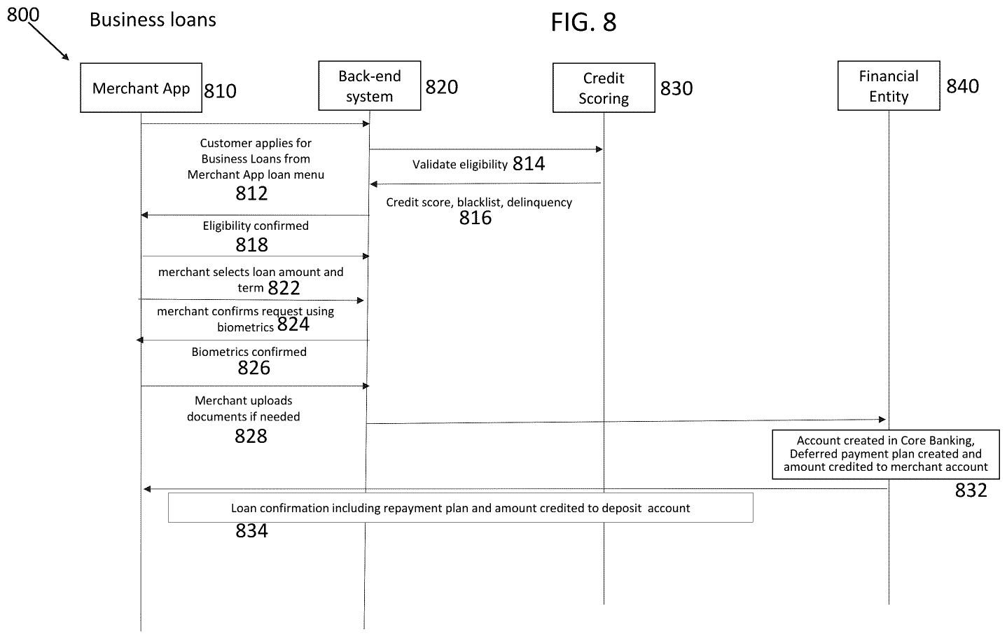

FIG. 8 is an example interactive logic flow 800 illustrating a method of providing access to business loans via merchant financing application, in accordance with an embodiment of this disclosure. Referring to FIG. 8, the interactive logic flow 800 includes a number of interaction stations, such as a customer application 810, a back-end system 820, a credit scoring agency 830, a financial entity 840 and the like. The “Business Loans” process begins when an option to apply for loans is displayed to the customer, who may be a merchant, on the home screen. Once clicked, all loans available to the customer are displayed on the screen, as in 812. One of the options on the loan menu may be “Business Loans”. Once the customer selects this option, as in 814, credit scores of the customer may be checked next to appraise the eligibility of the customer, as in 816 and as explained above in relation to the method of FIG. 2. After eligibility is confirmed (credit score of the customer being above a minimum threshold), as in 818, the insurance options are shared with the customer.

At this point, the merchant selects a loan amount and a term, as in 822. The merchant confirms the request using instant biometrics, as in 824. The instant biometrics may be compared with the ones submitted by the customer during onboarding, face or fingerprint depending on device and selection. Once the biometrics are approved, as in 826, there may be a request for additional documents, as in 828. Subsequently a loan account may be created in the core banking system, and/or ERP system and/or a CRM system, as in 832. A deferred payment plan may be created. Further, the customer may be able to view a summary of the loan application that includes the requested loan amount and the approved limit, instant biometrics. The instant biometrics may be compared with the ones submitted by the customer during onboarding, face or fingerprint depending on device and selection. Once the biometrics are approved, there may be a request for additional documents. Subsequently a loan account may be created in the core banking system, and/or ERP system and/or a CRM system. A deferred payment plan may be created. Further, the customer may be able to view a summary of the loan application that includes the requested loan amount and the approved limit. Once consented, the loan amount may be credited to the merchant account, as in 834. At this point, the customer may link a payment account and make necessary payments as explained above in relation to the method of FIG. 3.

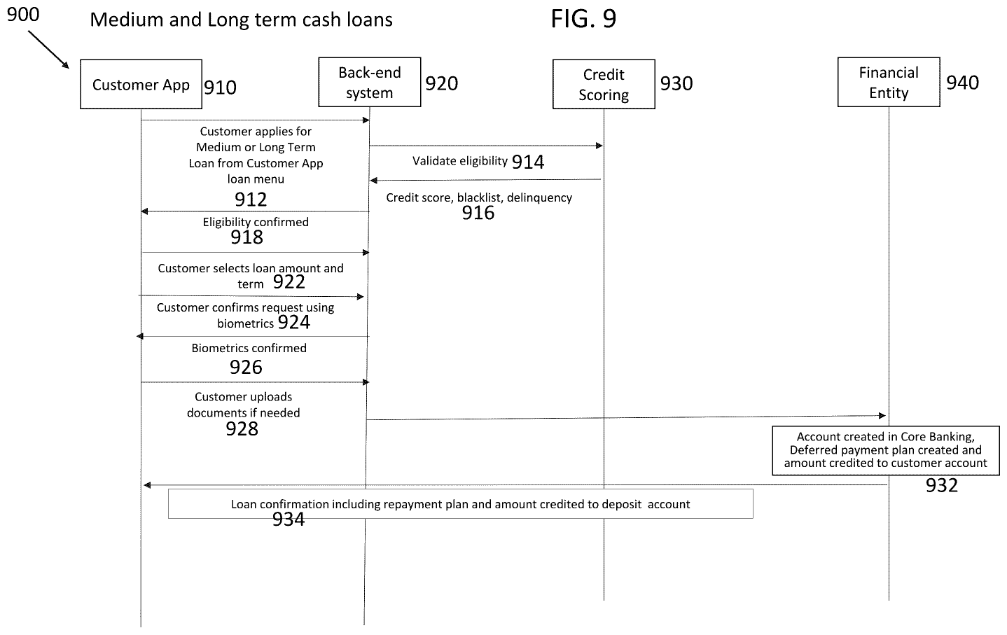

FIG. 9 is an example interactive logic flow 900 illustrating a method of providing access to medium-and-long-term cash loans via the consolidated communication and financial account of FIG. 1, in accordance with an embodiment of this disclosure. Referring to FIG. 9, the interactive logic flow 900 includes a number of interaction stations, such as a customer application 910, a back-end system 920, a credit scoring agency 930, a financial entity 940 and the like. The “Medium and Long Term Cash Loans” process begins when an option to apply for loans is displayed to the customer on the home screen. Once clicked, all loans available to the customer are displayed on the screen, as in 912. One of the options on the loan menu may be “Medium and Long term Cash Loans”. Once the customer selects this option, as in 914, credit scores of the customer may be checked next to appraise the eligibility of the customer, as in 916 and as explained above in relation to the method of FIG. 2. After eligibility is confirmed (credit score of the customer being above a minimum threshold), as in 918, the insurance options are shared with the customer.

At this point, the customer selects a loan amount and a term, as in 922. The customer confirms the request using instant biometrics, as in 924. The instant biometrics may be compared with the ones submitted by the customer during onboarding, face or fingerprint depending on device and selection. Once the biometrics are approved, as in 926, there may be a request for additional documents, as in 928. Subsequently a loan account may be created in the core banking system, and/or ERP system and/or a CRM system, as in 932. A deferred payment plan may be created. Further, the customer may be able to view a summary of the loan application that includes the requested loan amount and the approved limit. Once consented, the loan amount may be credited to the customer's account, as in 934. At this point, the customer may link a payment account and make necessary payments as explained above in relation to the method of FIG. 3.

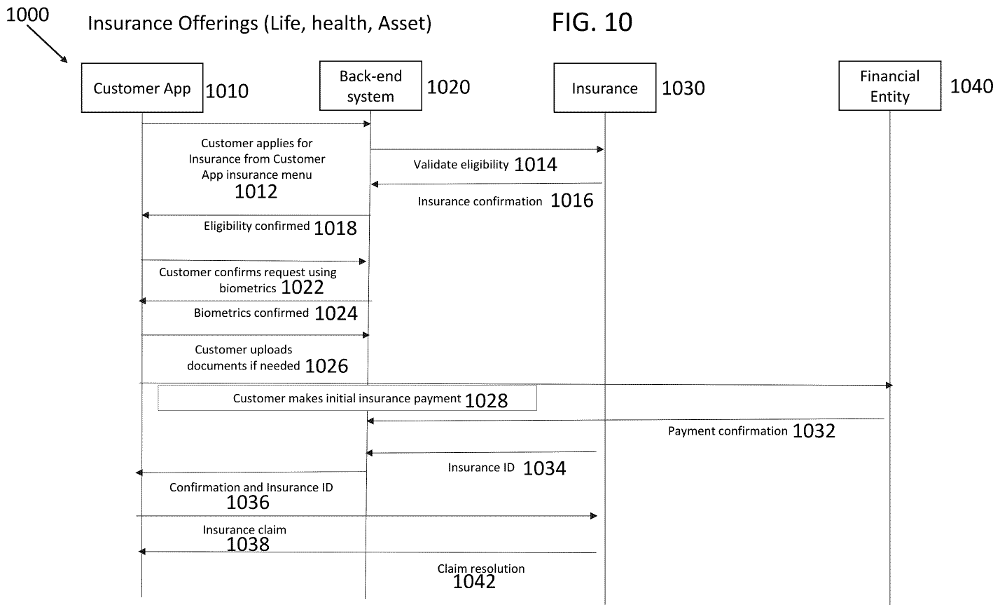

FIG. 10 is an example interactive logic flow 1000 illustrating a method of providing access to insurance offerings via the consolidated communication and financial account of FIG. 1, in accordance with an embodiment of this disclosure. Referring to FIG. 10, the interactive logic flow 1000 includes a number of interaction stations, such as a customer application 1010, a back-end system 1020, an insurance provider 1030, a financial entity 1040 and the like. The “Insurance Offerings” process begins when an option to apply for insurance is displayed to the customer on the home screen. Once clicked, all insurance offerings available to the customer are displayed on the screen, as in 1012. Once the customer selects an option, as in 1014, credit scores of the customer may be checked next to appraise the insurance eligibility of the customer, as in 1016 and as explained above in relation to the method of FIG. 2. After eligibility is confirmed (credit score of the customer being above a minimum threshold), as in 1018, the insurance options are shared with the customer.

At this point, the customer may select an insurance amount and a term. The customer confirms the request using instant biometrics, as in 1022. The instant biometrics may be compared with the ones submitted by the customer during onboarding, face or fingerprint depending on device and selection. Once the biometrics are approved, as in 1024, there may be a request for additional documents, as in 1026.

A first payment amount may be communicated to the customer and the customer may use the consolidated communication and financial account to make the payment, as in 1028. The back-end system may check for confirmation of the payment. Once the payment is confirmed, as in 1032, an account may be opened with an insurance provider and/or an ERP and/or a CRM system. A deferred payment plan may be created and an insurance ID may be provided to the customer, as in 1034. Customer may see a summary of the insurance application including all insurance details, as in 1036.

The customer may link a payment account to allow for payment of the insurance by via the consolidated communication and financial account. All transactions completed by the customer may get updated into the insurance account. The system may continuously monitor for timely payments and it is responsive to determining that the insurance hasn't been paid. In case of late payment, the credit score of the customer may be updated and the impact may be notified to the customer prompting for quick payment.

Further, insurance claims may be initiated, as in 1038, from the consumer based financial application without any need for any paperwork. All documents required by the insurer may be uploaded via the consumer based financial application and any claims may be sent directly to the insurer. The insurance provider may audit the claim and accept or reject the same. Once the claim is accepted and resolved, as in 1042, the resolution may be updated in the consumer based financial application and a payment may be deposited into the customer's account.

FIG. 11 is an example interactive logic flow 1100 illustrating a method of providing access to goal-based savings via the consolidated communication and financial account, in accordance with an embodiment of this disclosure. Referring to FIG. 11, the interactive logic flow 1100 includes a number of interaction stations, such as a customer application 1110, a back-end system 1120, an eligibility assessment system 1130, a financial entity 1140 and the like. The “Goal Based Savings” process begins when an option to apply for “Goal Based Savings” is displayed to the customer on the home screen. Once clicked, all different offerings available to the customer (such as, education, health and the like) are displayed on the screen, as in 1112. Once the customer selects this option, as in 1114, credit scores of the customer may be checked next to appraise the eligibility of the customer, as in 1116 and as explained above in relation to the method of FIG. 2. After eligibility is confirmed (credit score of the customer being above a minimum threshold), as in 1118, the goal-based savings options are shared with the customer.

At this point, the customer may subscribe, as in 1122 and make the initial savings payment, as in 1124. A savings account may be created in a core banking system and/or an ERP system and/or a CRM system, as in 1126. A confirmation message may be shared with the customer in the consolidated communication and financial account. The savings account balance may be part of the customer's products on the home page. There may also be a tracker that display the balance remaining to achieve the savings goal, as in 1128. Further, responsive to successful receipt of a minimum number of deposits, the system may automatically offer a subsequent (second or third and so forth) goal to the customer. The terms of the subsequent goals may be based on a history of the customer's account usage and deposits.

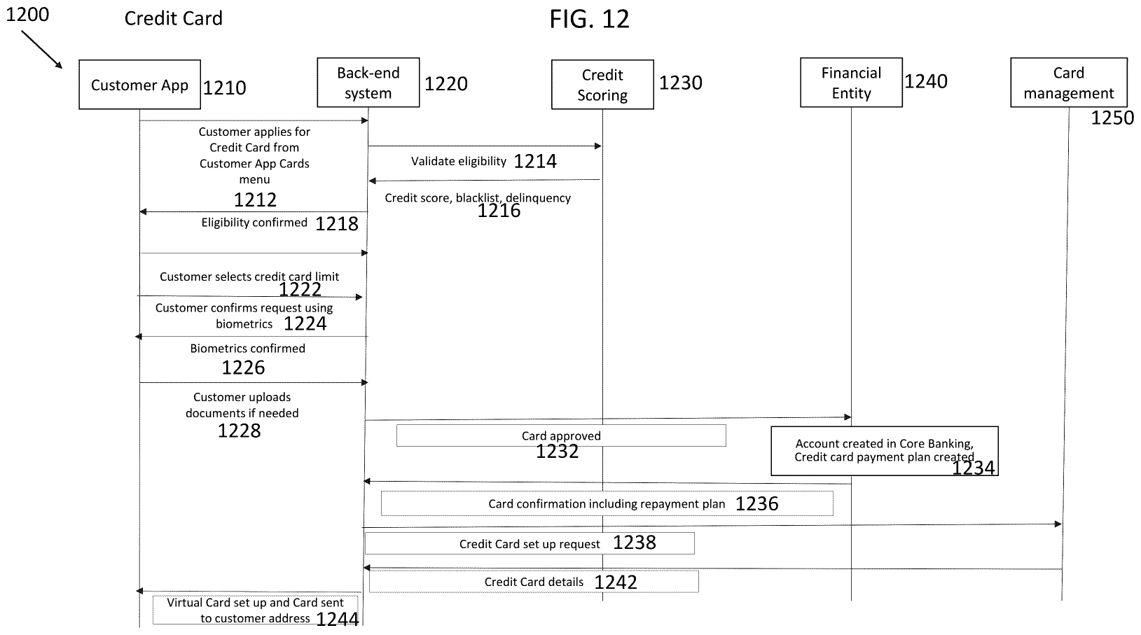

FIG. 12 is an example interactive logic flow 1200 illustrating a method of providing access to credit cards via the consolidated communication and financial account of FIG. 1, in accordance with an embodiment of this disclosure. Referring to FIG. 12, the interactive logic flow 1200 includes a number of interaction stations, such as a customer application 1210, a back-end system 1220, a credit scoring agency 1230, a financial entity 1240, a card management organization 1250 and the like. The “Credit Card Procurement” process begins when an option to apply for credit card displayed to the customer on the home screen. Once clicked, all credit card options available to the customer are displayed on the screen, as in 1212. Once the customer selects an option, as in 1214, credit scores of the customer may be checked next to appraise the eligibility of the customer, as in 1216 and as explained above in relation to the method of FIG. 2. After eligibility is confirmed (credit score of the customer being above a minimum threshold), as in 1218, the credit card options are shared with the customer.

At this point, the customer selects a credit card limit, as in 1222. The customer confirms the request using instant biometrics, as in 1224. The instant biometrics may be compared with the ones submitted by the customer during onboarding, face or fingerprint depending on device and selection. Once biometrics are approved, as in 1226, there may be a request for additional documents if required, as in 1228. The customer may be able to view a summary of the card application on the home page of the consolidated communication and financial account, under “Cards” tab. The customer may also view the status of the card application, the requested amount. If approved, as in 1232, a credit account may be created in the core banking system, and/or ERP system and/or a CRM system and/or a card management system, as in 1234. A deferred payment plan may be created, as in 1236. A credit card may be assigned to the customer, as in 1238 and may be sent to the customer address, as in 1242. The credit card may also be available as a virtual card for payments using the app, as in 1244. At this point, the customer may link a payment account and make necessary payments as explained above in relation to the method of FIG. 3.

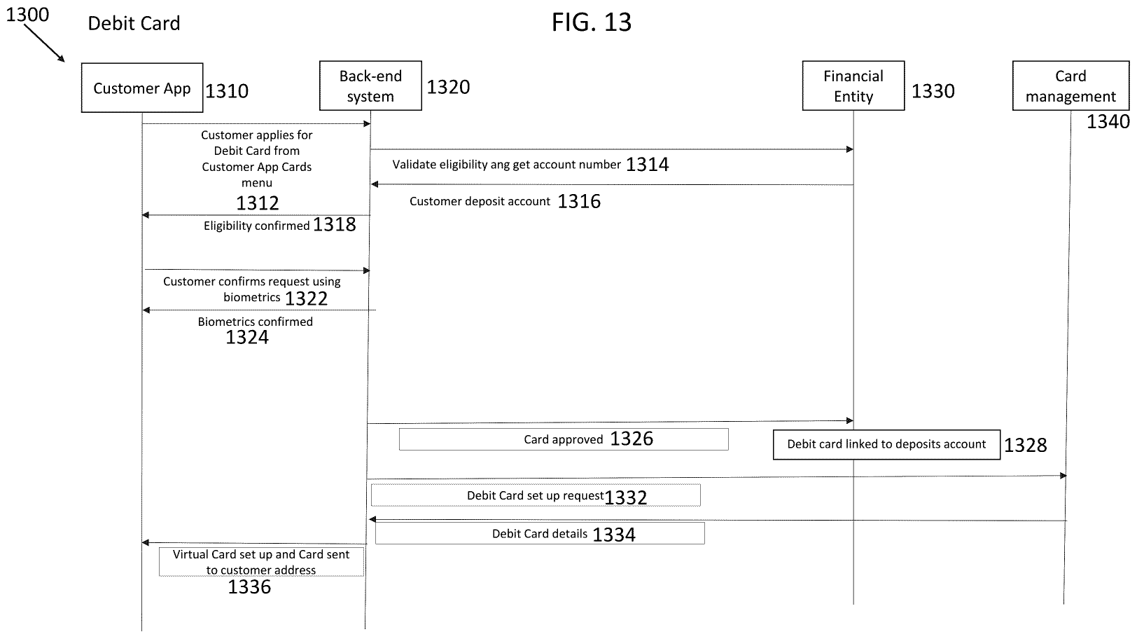

FIG. 13 is an example interactive logic flow 1300 illustrating a method of providing access to debit cards via the consolidated communication and financial account of FIG. 1, in accordance with an embodiment of this disclosure. Referring to FIG. 13, the interactive logic flow 1300 includes a number of interaction stations, such as a customer application 1310, a back-end system 1320, a financial entity 1330, a card management organization 1340 and the like. The “Debit Card Procurement” process begins when an option to apply for a debit card is displayed to the customer on the home screen. Once clicked, all card options available to the Customer are displayed, as in 1312. There may be an option to apply for the selected Debit Card. Once the customer selects a debit card option, as in 1314, eligibility check may be completed, as in 1316. The customer needs to have a savings, a wallet or other deposit account to be qualified. A customer account number is provided along with eligibility approval, as in 1318. The customer confirms the request using instant biometrics, as in 1322. The instant biometrics may be compared with the ones submitted by the customer during onboarding, face or fingerprint depending on device and selection. Once biometrics are approved, as in 1324, the customer may see a summary of the debit card application, as in 1326. A debit card may be linked to the selected account, as in 1328. The debit card account may also be created in the card management system, as in 1332. A debit card may be assigned to the customer and sent to the customer's address, as in 1334. The debit card may also be available as a virtual card for payments using the application, as in 1336.

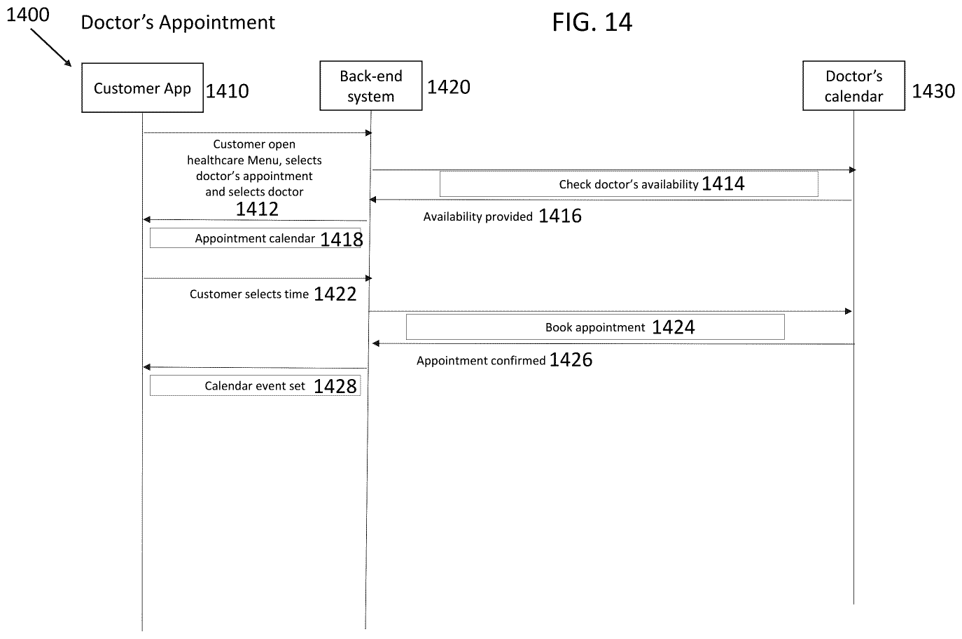

FIG. 14 is an example interactive logic flow 1400 illustrating a method of providing access to doctors' appointments via the consolidated communication and financial account of FIG. 1, in accordance with an embodiment of this disclosure. Referring to FIG. 14, the interactive logic flow 1400 includes a number of interaction stations, such as a customer application 1410, a back-end system 1420, a Doctor's calendar 1430) and the like. The “Doctor's Appointment” process begins when an option to enter “Healthcare” menu is displayed to the customer on the home screen. Once clicked, all healthcare offerings available to the customer are displayed on the screen, as in 1412. The customer selects the “Doctor's Appointment” option and selects a doctor. The doctor's availability is checked, as in 1414, using the doctor's calendar which is available via APIs calls to the back-end of the system. In effect, availability information of a doctor is provided to the back-end of the system, as in 1416 and then to the customer, as in 1418. After getting a confirmation from customer, as in 1422, the back-end system reserves an appointment on the doctor's calendar, as in 1424 and the appointment is confirmed to the customer, as in 1426. At this point, the doctor's appointment is scheduled on the customer's calendar, as in 1428.

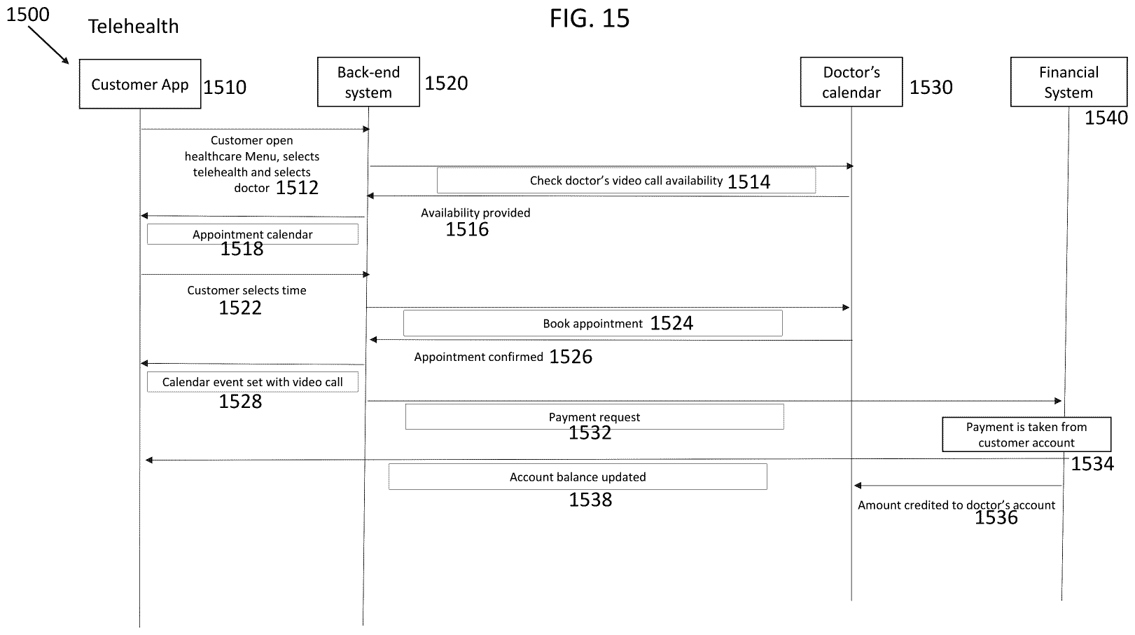

FIG. 15 is an example interactive logic flow 1500 illustrating a method of providing access to telehealth via the consolidated communication and financial account of FIG. 1, in accordance with an embodiment of this disclosure. Referring to FIG. 15, the interactive logic flow 1500 includes a number of interaction stations, such as a customer application 1510, a back-end system 1520, a Doctor's calendar 1530, a financial system 1540 and the like. The “Telehealth” process begins when an option to enter healthcare menu is displayed to the customer on the home screen. Once clicked, all healthcare offerings available to the customer are displayed on the screen, as in 1512. The customer selects the “Telehealth” option and selects a doctor. The doctor's video availability is checked, as in 1514, using the doctor's calendar which is available via APIs calls to the back-end of the system. In effect, availability information of a doctor is provided to the back-end of the system, as in 1516 and then to the customer, as in 1518. After getting a confirmation from the customer, as in 1522, the back-end system reserves a telehealth appointment on the doctor's calendar, as in 1524 and the appointment is confirmed to the customer, as in 1526. A Telehealth calendar appointment is scheduled on the customer calendar including video call, as in 1528. The customer may be informed about an amount of payment and a payment request may be automatically generated from the customer's account, as in 1532. The payment amount may be automatically withdrawn from the customer's account, as in 1534. The amount may be credited to the doctor's account, as in 1536 and the customer's account balance may be updated, as in 1538.

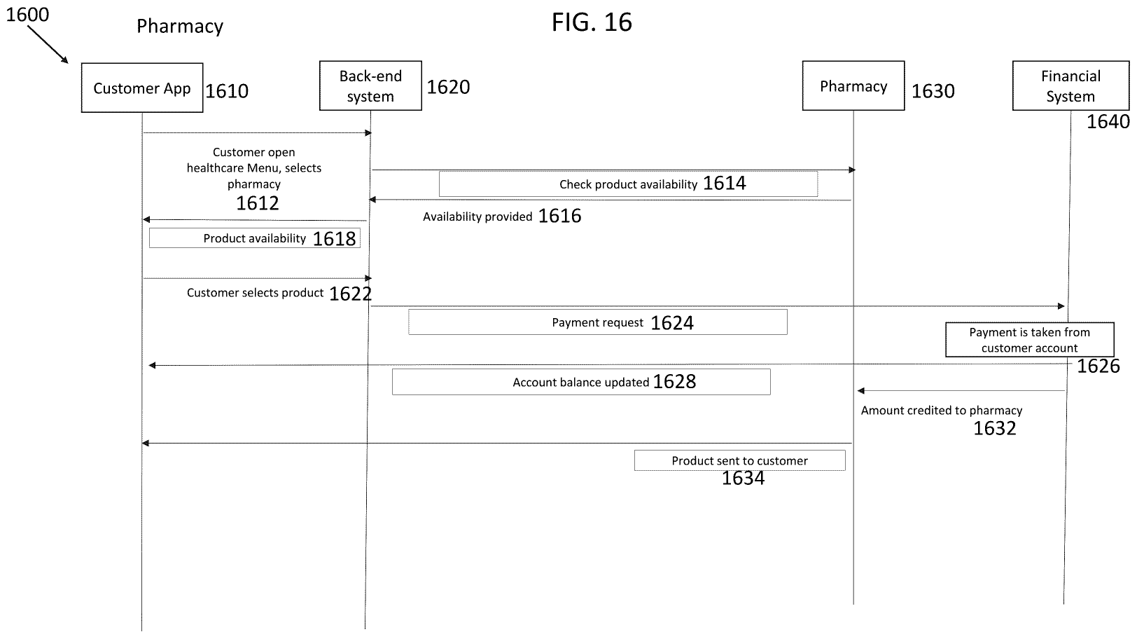

FIG. 16 is an example interactive logic flow 1600 illustrating a method of providing access to pharmacy via the consolidated communication and financial account of FIG. 1, in accordance with an embodiment of this disclosure. Referring to FIG. 16, the interactive logic flow 1600 includes a number of interaction stations, such as a customer application 1610, a back-end system 1620, a pharmacy 1630, a financial system 1640 and the like. The “Pharmacy” process begins when an option to enter healthcare menu is displayed to the customer on the home screen. Once clicked, all healthcare offerings available to the customer are displayed on the screen, as in 1612. The customer may select the “Pharmacy” option and search and select a product. The product's availability may be checked, as in 1614, using the pharmacy's database which is available via APIs to the back-end of the system. In effect, availability information of the product is provided to the back-end of the system, as in 1616 and then to the customer, as in 1618. After getting a confirmation from the customer, as in 1622, the back-end system communicates a product request to the pharmacy. The customer may be informed about an amount of payment and a payment request may be automatically generated from the customer's account, as in 1624. The payment amount may be automatically withdrawn from the customer's account, as in 1626. The customer's account balance is updated and the amount is credited to the pharmacy, as in 1632. The product is shipped to the customer's address, as in 1634.

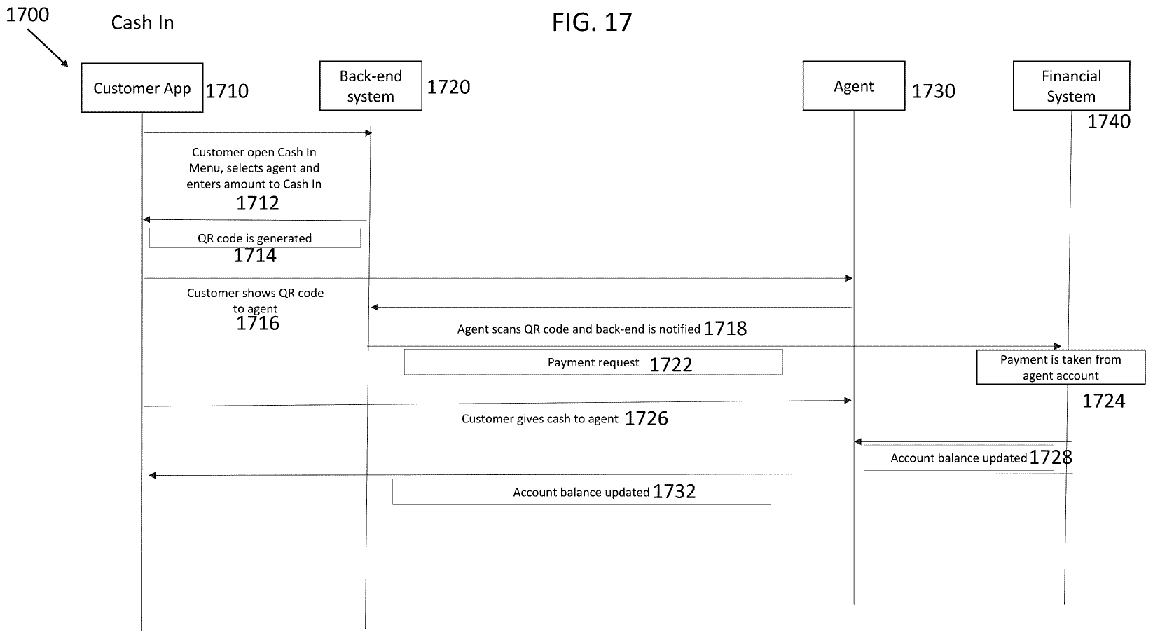

FIG. 17 is an example interactive logic flow 1700 illustrating a method of providing access to cash-in financing via the consolidated communication and financial account of FIG. 1, in accordance with an embodiment of this disclosure. Referring to FIG. 17, the interactive logic flow 1700 includes a number of interaction stations, such as a customer application 1710, a back-end system 1720, an agent 1730, a financial system 1740 and the like. The “Cash In” process begins when a “Cash In” option is displayed to the customer on the home screen. Once clicked, all agents available to the customer are displayed on the screen. The customer selects an agent and enters an amount of cash that is required, as in 1712. A QR code with the amount is generated and made available in the application to be used when reaching out to the agent, as in 1714. The customer is shown an “Agents Nearby’ option. At this point, the screen displays a list of agents, their contact details, and the distance between the customer's location and the agents” locations. Once an agent is selected, directions to the agent may be displayed using the location capabilities of the device and the “map” application. The customer may find an agent to access the cash-in option. The customer may provide the QR code to the agent, as in 1716 and the agent may scan the QR code and thereby notify the back-end system, as in 1718. The back-end system may send the agent a notification that informs an amount of payment. A payment request is automatically generated from the agent's account, as in 1722. At this point, the payment amount may be automatically withdrawn from the customer's account, as in 1724. In another case, the customer may hand over the cash to the agent, as in 1726. The agent's account balance may be updated, as in 1728 and the customer's account balance may be updated, as in 1732.

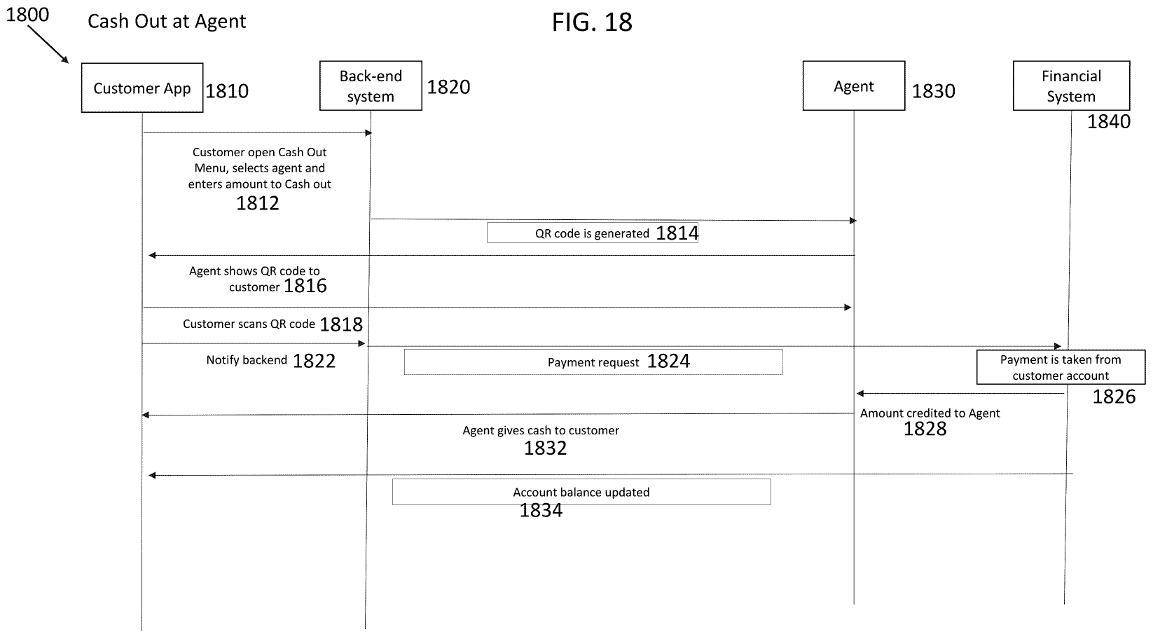

FIG. 18 is an example interactive logic flow 1800 illustrating a method of providing access to cash-out-at-agent financing via the consolidated communication and financial account of FIG. 1, in accordance with an embodiment of this disclosure. Referring to FIG. 18, the interactive logic flow 1800 includes a number of interaction stations, such as a customer application 1810, a back-end system 1820, an agent 1830, a financial system 1840) and the like. The “Cash Out at Agent” process begins when a “Cash Out at Agent” option is displayed to the customer on the home screen. Once clicked, all agents available to the customer may be displayed on the screen, as in 1812. The customer may select an agent and enter an amount of cash that is required. A QR code with the amount may be generated, as in 1814 and made available in the application to be used when reaching out to an agent, as in 1816. The customer is shown an “Agents Nearby” option. At this point, the screen displays a list of agents, their contact details, and the distance between the customer's location and the agent's location. Once an agent is selected, directions to the agent may be displayed using the location capabilities of the device and the “maps” application. The customer may find an agent to access the cash-out option. The agent may provide the QR code to the customer and the customer may scan the QR code, as in 1818 and thereby notify the back-end system, as in 1822. The customer may be informed about an amount of payment and a payment request may be sent, as in 1824. The payment amount may be automatically withdrawn from the customer's account, as in 1826 and the amount may get credited to the agent's account, as in 1828. At this point, the agent may hand over the cash to the customer, as in 1832. The customer's account balance and the agent's account balance may be updated, as in 1834.

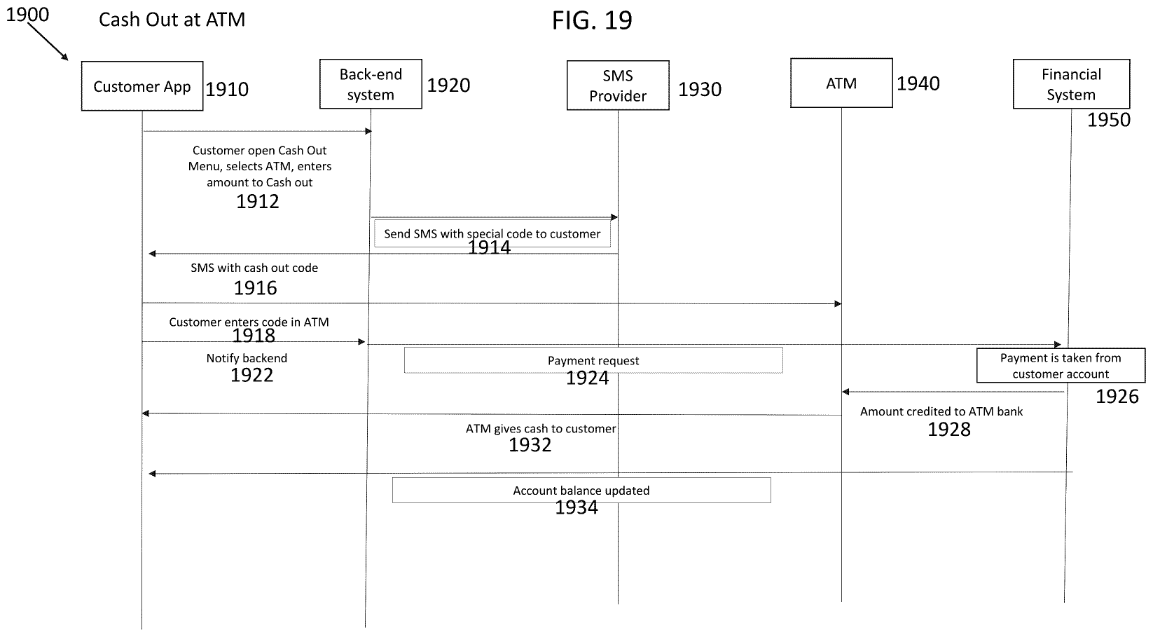

FIG. 19 is an example interactive logic flow 1900 illustrating a method of providing access to cash-out-at-ATM financing via the consolidated communication and financial account of FIG. 1, in accordance with an embodiment of this disclosure. Referring to FIG. 19, the interactive logic flow 1900 includes a number of interaction stations, such as a customer application 1910, a back-end system 1920, an SMS provider 1930, an ATM 1940, a financial system 1950 and the like. The “Cash Out at ATM” process begins when a “Cash Out at ATM” option is displayed to the customer on the home screen. Once clicked, all ATMs available to the Customer may be displayed on the screen, as in 1912. The customer may select an ATM and enter an amount of cash that is required. A “Cash Out” code with the amount may be generated, as in 1914 and sent to the customer via an SMS, as in 1916 which may be available in the application and to be used when accessing an ATM.

The customer may be shown a “ATMs Nearby” option. At this point, the screen may display a list of ATMs, and the distance between the customer's location and the ATMs' locations. Once an ATM is selected, directions to the ATM may be displayed using the location capabilities of the device and the “maps” application. The customer may find an ATM to perform the “Cash Out” operation. The customer may enter the “Cash Out” code in the ATM, as in 1918 and thereby notify the back-end system, as in 1922. The customer may be informed about an amount of payment and a payment request may be automatically generated from the customer's account, as in 1924. The amount may be automatically withdrawn from the customer's account, as in 1926 and credited into the ATM Bank's account, as in 1928. The ATM may dispense the cash to the customer, as in 1932. The customer's account balance and the agent's account balance may be updated, as in 1934.

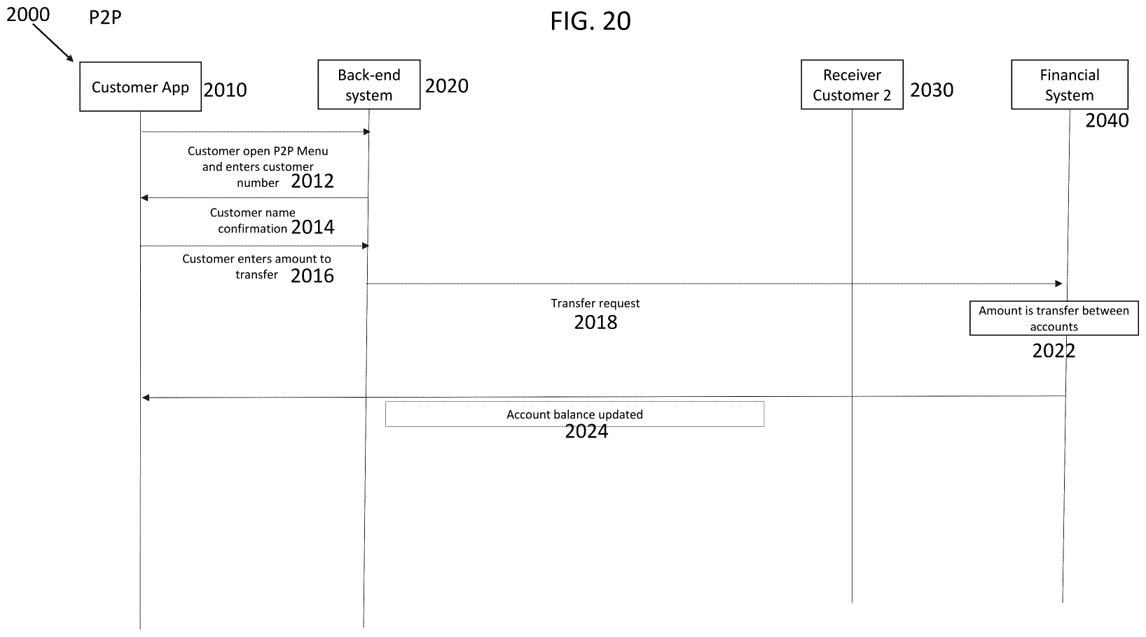

FIG. 20 is an example interactive logic flow 2000 illustrating a method of providing access to peer-to-peer (P2P) financing via the consolidated communication and financial account of FIG. 1, in accordance with an embodiment of this disclosure. Referring to FIG. 20, the interactive logic flow 2000 includes a number of interaction stations, such as a customer application 2010, a back-end system 2020, receiver customers 2030, a financial system 2040 and the like. The “Peer to Peer Transfer” process begins when an option to perform P2P transfers is displayed to the customer on the home screen, as in 2012. Once clicked, the screen display how to transfer to P2P accounts, as in 2014. The customer may enter the phone number of another transferee customer, as in 2016. Further, the customer may confirm the receiver and enter an amount to be transferred, as in 2018. The customer may be informed about the transferee and the amount may be automatically withdrawn from the customer's account, as in 2022 and credited into the receiver's account. The customer account balance and the receiver's account balance may be updated, as in 2024.

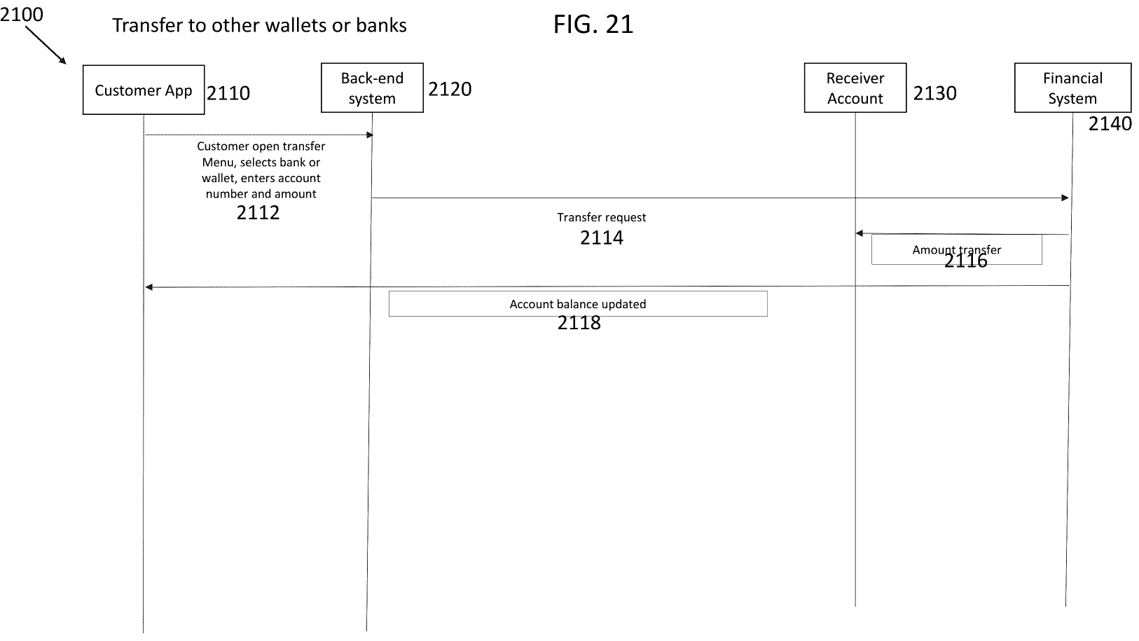

FIG. 21 is an example interactive logic flow 2100 illustrating a method of providing access to money transfer service via the consolidated communication and financial account of FIG. 1, in accordance with an embodiment of this disclosure. Referring to FIG. 21, the interactive logic flow 2100 includes a number of interaction stations, such as a customer application 2110, a back-end system 2120, receiver accounts 2130, a financial system 2140) and the like. The “Transfers to other wallet or banks” process begins when an option to perform money transfers to other accounts is displayed on the screen. Once clicked, the system may display how to transfer to other accounts, as in 2112. The customer may select the bank or wallet where the recipient account is. The customer may enter the account number information of the transferee account. The customer may confirm the details of the transferee account and enter the amount to be transferred, as in 2114. The amount may be automatically withdrawn from the customer's account, as in 2116 and the transferee account may get credited. The customer's account balance and the receiver's account balance may be updated, as in 2118.

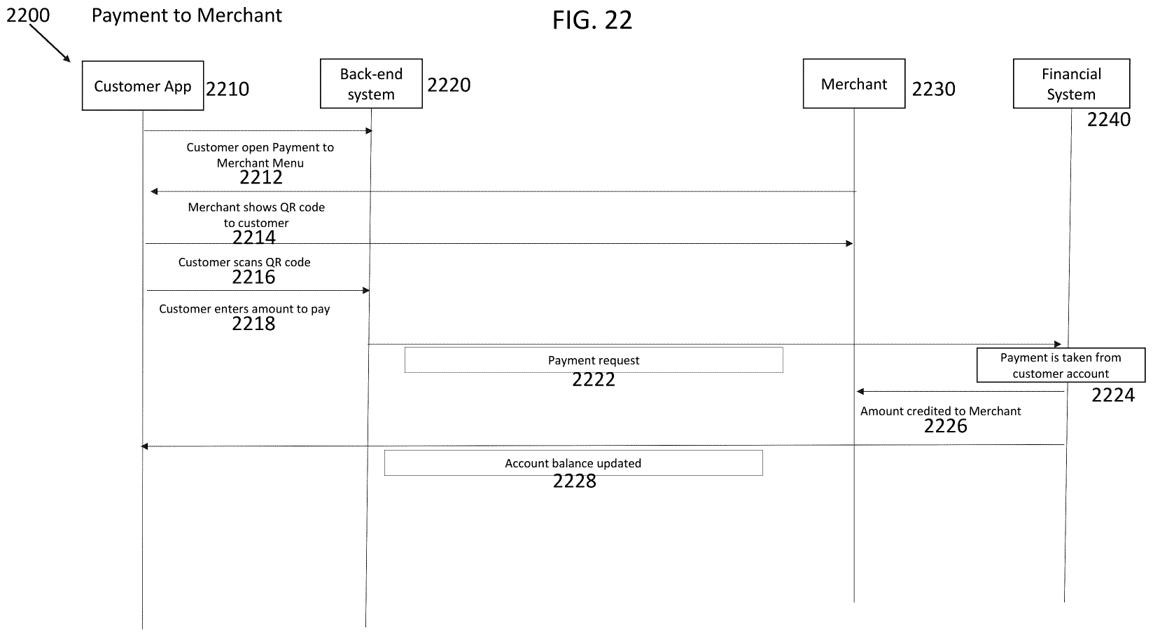

FIG. 22 is an example interactive logic flow 2200 illustrating a method of providing access to merchant payments via the consolidated communication and financial account of FIG. 1, in accordance with an embodiment of this disclosure. Referring to FIG. 22, the interactive logic flow 2200 includes a number of interaction stations, such as a customer application 2210, a back-end system 2220, a merchant 2230, a financial system 2240 and the like. The “Payments to Merchants” process begins when an option to perform “Payments to Merchants” is displayed on the screen. Once clicked, the system may display how to make payments to a merchant, as in 2212. The merchant 2230 may display a QR code for scanning by the customer application 2210, as in 2214. The customer may scan the QR code, as in 2216 and enter an amount to pay, as in 2218. A payment request may be sent, as in 2222. The payment may be automatically withdrawn from the customer's account, as in 2224 and the merchant's account may get credited, as in 2226. The customer's account balance and the merchant's account balance may be updated, as in 2228.

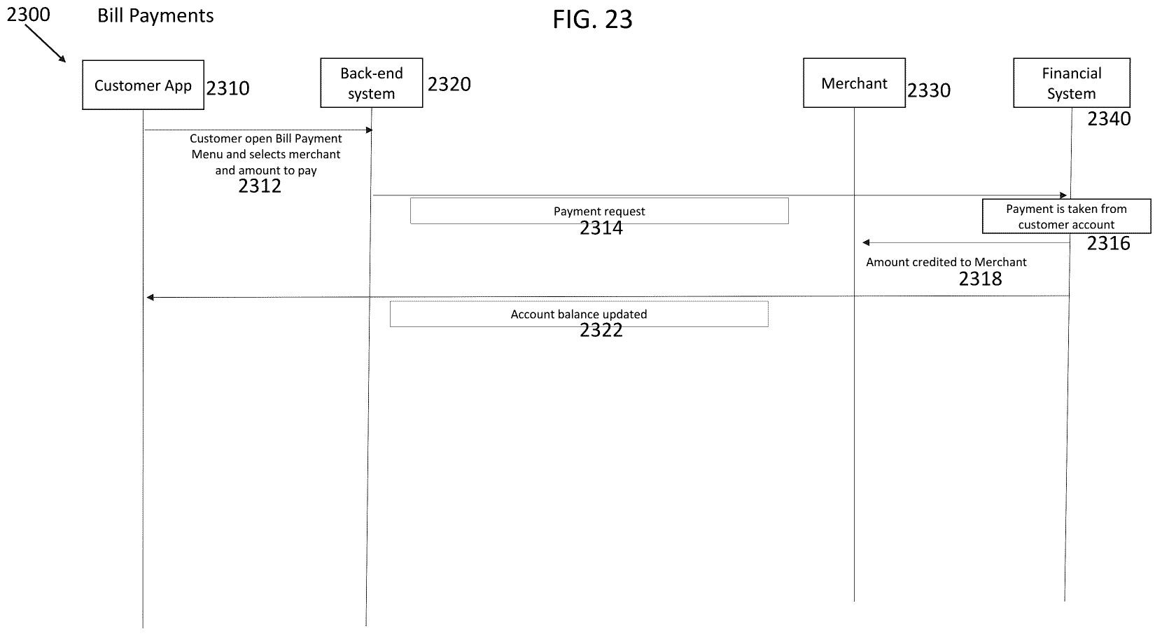

FIG. 23 is an example interactive logic flow 2300 illustrating a method of providing access to bill payments via the consolidated communication and financial account of FIG. 1, in accordance with an embodiment of this disclosure. Referring to FIG. 23, the interactive logic flow 2300 includes a number of interaction stations, such as a customer application 2310, a back-end system 2320, a merchant 2330, a financial system 2340) and the like. The “Bill Payments” process begins when a “Bill Payments” option is displayed on the screen. Once clicked, the system may display instruction on how to make Bill Payments to utility providers, as in 2312. The customer may select a particular payee utility from a list of utilities (such as electricity, water and the like). The customer may enter an account number and an amount to pay, as in 2314. The payment may be automatically withdrawn from the customer's account, as in 2316 and an account of the utility provider may get credited, as in 2318. The customer's account balance and the merchant's account balance are updated, as in 2322.

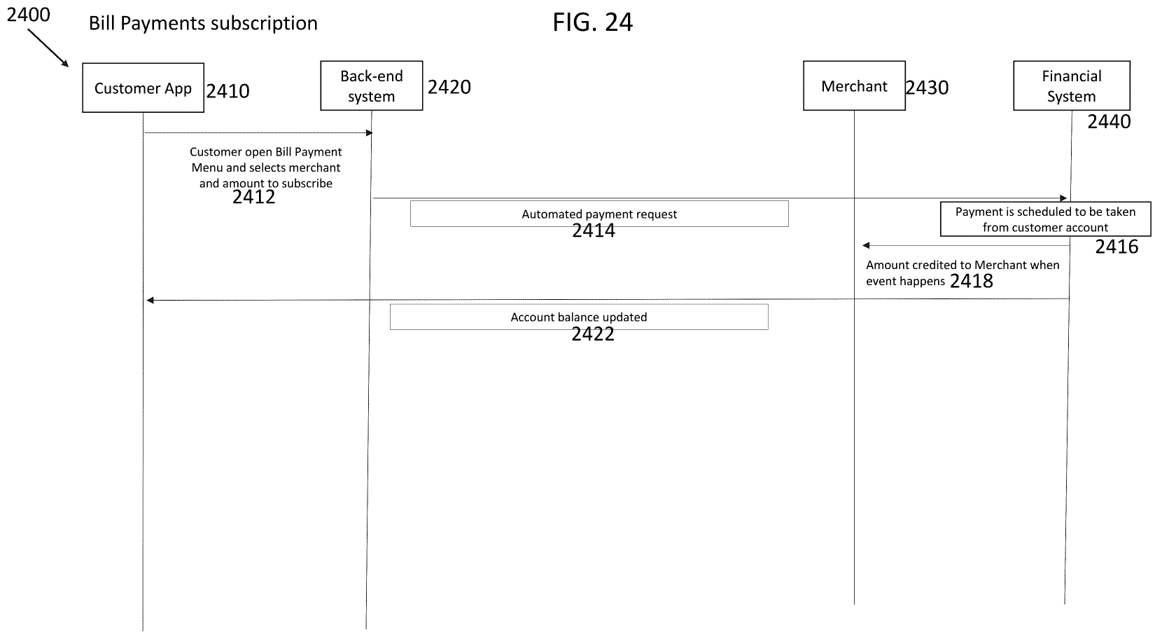

FIG. 24 is an example interactive logic flow 2400 illustrating a method of providing access to bill payments subscription via the consolidated communication and financial account of FIG. 1, in accordance with an embodiment of this disclosure. Referring to FIG. 24, the interactive logic flow 2400 includes a number of interaction stations, such as a customer application 2410, a back-end system 2420, a merchant 2430), a financial system 2440) and the like. The “Bill Payments Subscription” process begins when a “Bill Payments Subscription” option is displayed on the screen. Once clicked, the system may display instructions on how to make bill payments subscriptions to utility providers, as in 2412. The customer may select a particular payee utility from a list of utilities (such as electricity, water and the like). The customer may enter an account number and an amount to pay, as in 2414. The customer may subscribe to automated payment and the amount may be deducted from the customer's account, as in 2416 on a specified due date. The payment may be automatically withdrawn from the customer's account and an account of the utility provider may get credited, as in 2418. The customer's account balance and the merchant's account balance are updated, as in 2422.

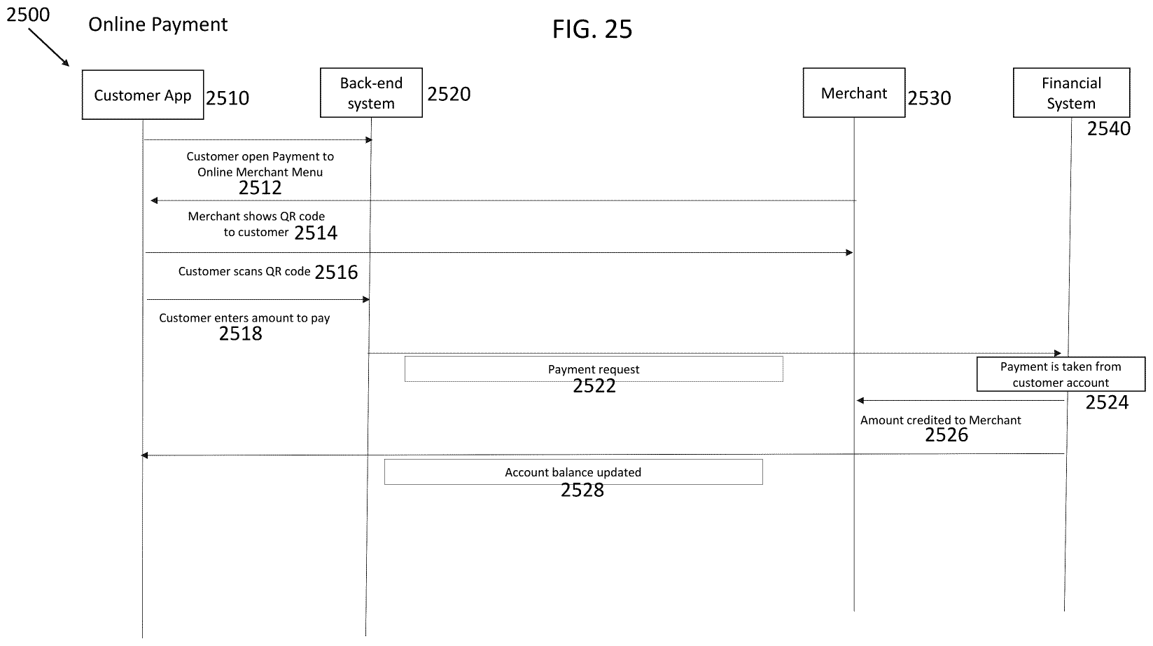

FIG. 25 is an example interactive logic flow 2500 illustrating a method of providing access to online payments via the consolidated communication and financial account of FIG. 1, in accordance with an embodiment of this disclosure. Referring to FIG. 25, the interactive logic flow 2500 includes a number of interaction stations, such as a customer application 2510, a back-end system 2520, a merchant 2530, a financial system 2540 and the like. The “Online Payment” process begins when a “Payment to Online Merchant” option is displayed on the screen. Once clicked, the system may display instructions on how to make online payments to merchants, as in 2512. The merchant 2530 may display a QR code for scanning by the customer application 2510. The customer may scan the QR code of the payee online merchant, as in 2516, enter an amount to pay, as in 2518, and send a payment request, as in 2522. The payment amount may be automatically withdrawn from the customer's account, as in 2524 and the merchant's account may get credited, as in 2526. The customer's account balance and the merchant's account balance may be updated, as in 2528. Further, there may be options in online stores to select the consolidated communication and financial account, as a method of payment. If selected, the system may open the consolidated communication and financial account and the customer may be directed to proceed with the payment for the selected merchant.

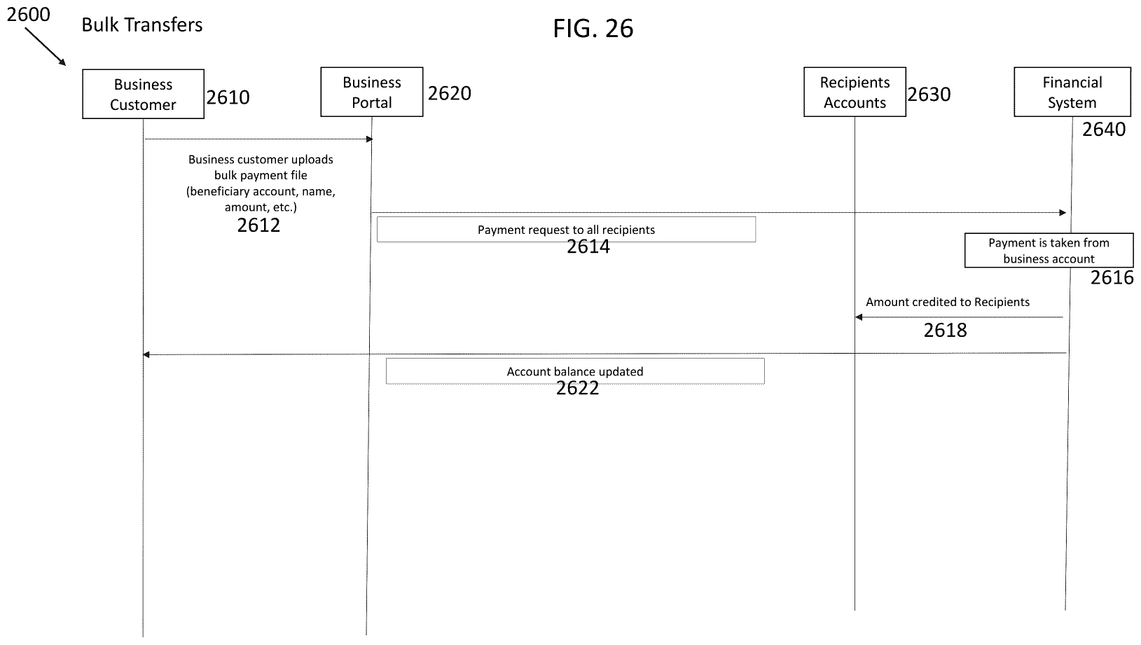

FIG. 26 is an example interactive logic flow 2600 illustrating a method of providing access to bulk payment service offerings via the consolidated communication and financial account of FIG. 1, in accordance with an embodiment of this disclosure. Bulk Payment Service offerings are convenient, cost-effective and secure ways for Business Customers seeking to process large volumes of payments and transfers at any one time. These offerings may be used for payroll or supplier payment. Referring to FIG. 26, the interactive logic flow 2600 includes a number of interaction stations, such as a customer application 2610, a back-end system 2620, recipients' accounts 2630, a financial system 2640 and the like. The “Bulk Transfers” process begins when a business customer uploads bulk payment file (a list of beneficiary accounts, names, amounts etc.) to a business portal, as in 2612. A payment request may to be sent to all recipients via the financial system 2640, as in 2614. The payment amount may be automatically withdrawn from the business account, as in 2616. The amount may be automatically sent to all recipients and credited to their accounts, as in 2618. The customer's account balance and the merchant's account balance may be updated, as in 2622.

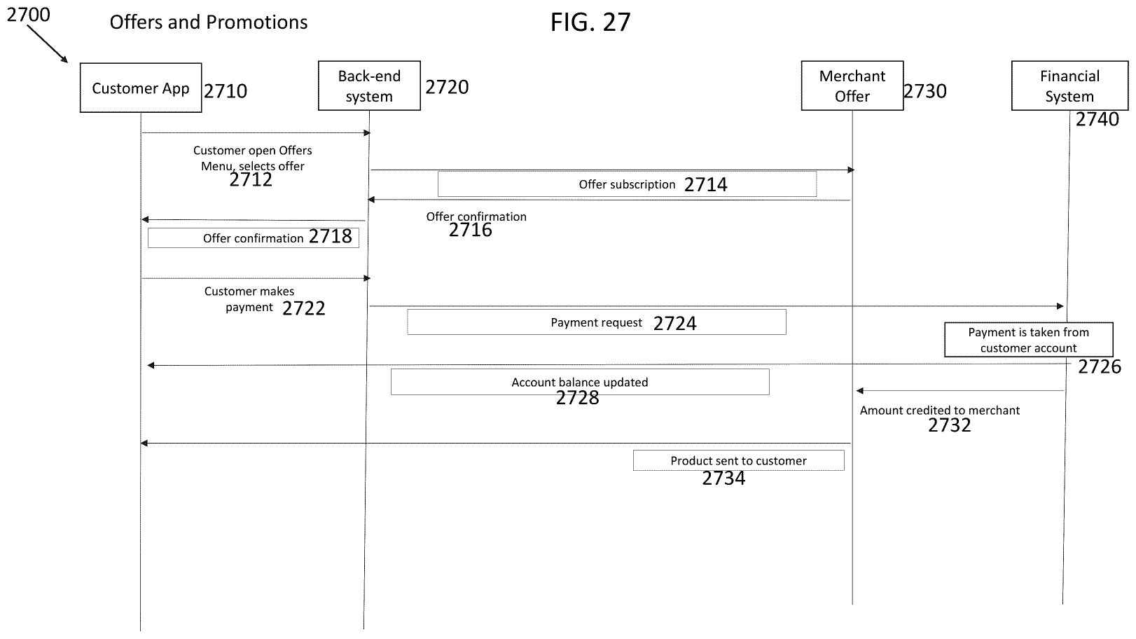

FIG. 27 is an example interactive logic flow 2700 illustrating a method of providing access to offers and promotions via the consolidated communication and financial account of FIG. 1, in accordance with an embodiment of this disclosure. Referring to FIG. 27, the interactive logic flow 2700 includes a number of interaction stations, such as a customer application 2710, a back-end system 2720, a merchant offer 2730, a financial system 2740) and the like. The “Offer and Promotions” process begins when an option to select “Offer and Promotions” is displayed to a customer. Once clicked, the system may display instructions on how to subscribe to a merchant's offer, as in 2712. The customer may subscribe to a particular offer, as in 2714 and receive confirmation from the merchant to the backend system 2720, as in 2716 and to the customer application 2710, as in 2718. The customer may enter an amount to pay, as in 2722 and send a payment request, as in 2724. The payment amount may be automatically withdrawn from the customer's account, as in 2726. The customer's account balance and the merchant's account balance may be updated, as in 2728. The merchant's account may get credited, as in 2732 and the product may be shipped to the customer, as in 2734.

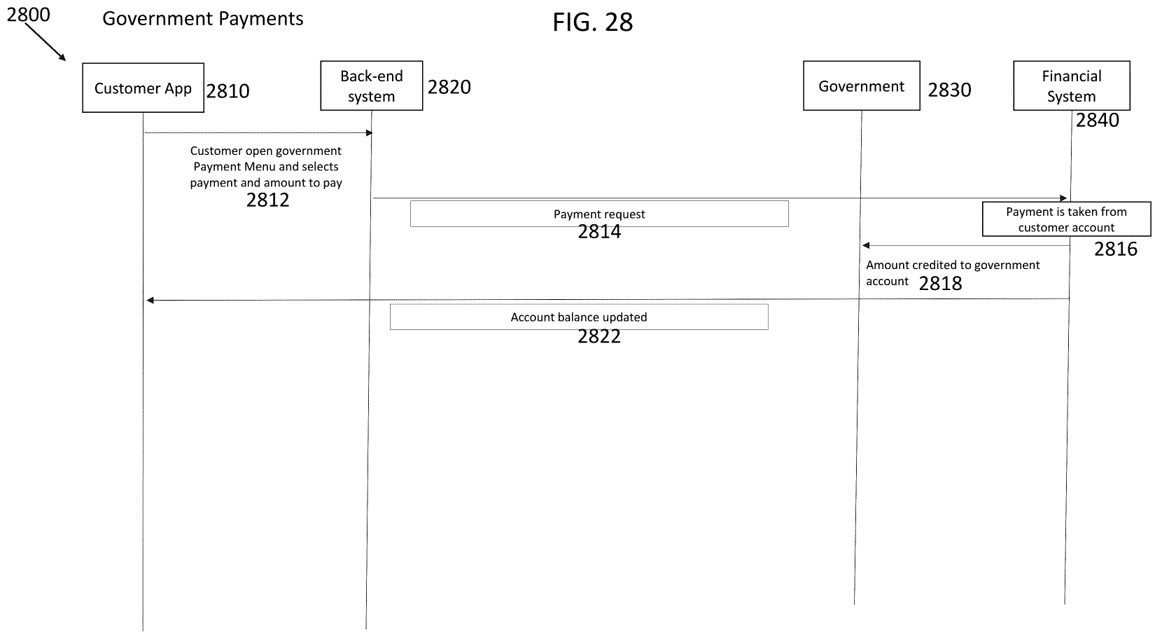

FIG. 28 is an example interactive logic flow 2800 illustrating a method of providing access to government payments via the consolidated communication and financial account of FIG. 1, in accordance with an embodiment of this disclosure. Referring to FIG. 28, the interactive logic flow 2800 includes a number of interaction stations, such as a customer application 2810, a back-end system 2820, a Government website 2830, a financial system 2840 and the like. The “Government Payment” process begins when a “Government Payment” option is displayed on the screen. Once clicked, the system displays instructions on how to make government payments, as in 2812. The customer selects the payments that are to be made (such as, taxes, municipality charges and the like). The customer may enter an account number and an amount to pay, as in 2814. The payment amount may be automatically withdrawn from the customer's account, as in 2816 and the government account may get credited, as in 2818. The customer's account balance and the government account balance may be updated, as in 2822. The customer may also subscribe to automated payment and in that case, the amount may be deducted on a specified due date.

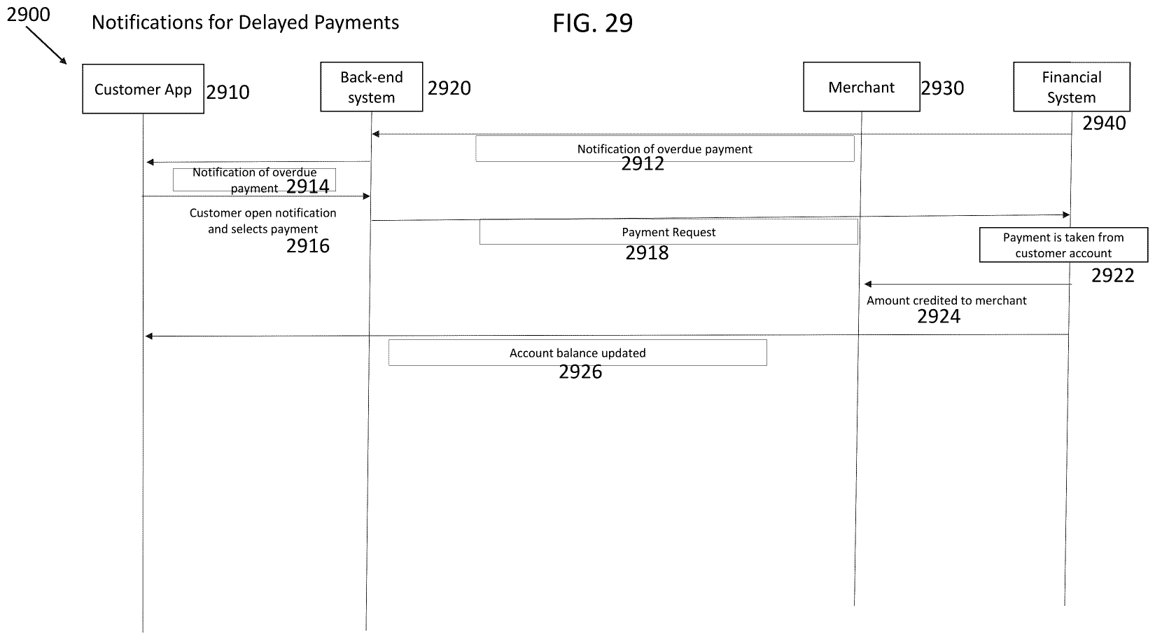

FIG. 29 is an example interactive logic flow 2900 illustrating a method of providing access to notifications for delayed payments via the consolidated communication and financial account of FIG. 1, in accordance with an embodiment of this disclosure. The consolidated communication and financial account may continuously monitor a customer's time-bound payments and may be responsive to determining that a payment is overdue or delayed. Referring to FIG. 29, the interactive logic flow 2900 includes a number of interaction stations, such as a customer application 2910, a back-end system 2920, a merchant 2930, a financial system 2940) and the like. The “Notifications for Delayed Payments” process begins when notifications for overdue payment are sent to the back system 2920, as in 2912 and then to the customer application 2910, as in 2914. The customer may open the notification and select a “Make Payment” option, as in 2916. The customer may enter an amount to pay and make a payment request, as in 2918. The payment amount may be automatically withdrawn from the customer's account, as in 2922 and the merchant's account may get credited, as in 2924. The customer's account balance and the merchant's account balance may be updated, as in 2926.

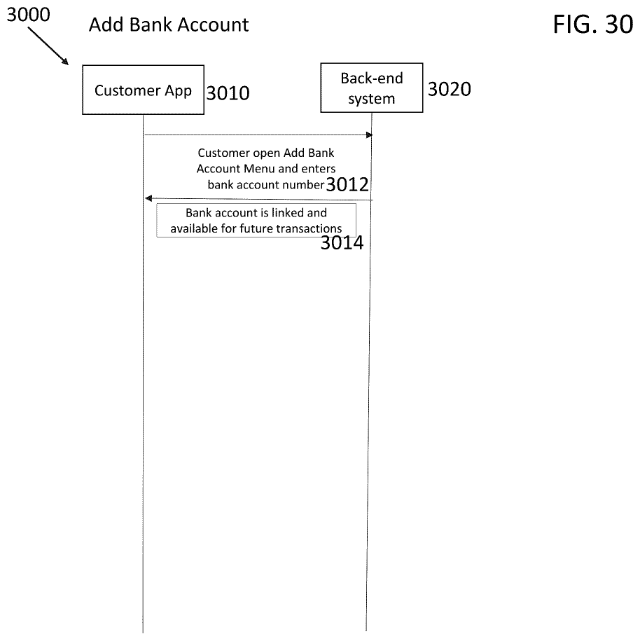

FIG. 30 is an example interactive logic flow 3000 illustrating a method of providing access to add bank accounts via the consolidated communication and financial account of FIG. 1, in accordance with an embodiment of this disclosure. Referring to FIG. 30, the interactive logic flow 3000 includes a number of interaction stations, such as a customer application 3010, a back-end system 3020 and the like. The “Add a bank account” process begins when a “Add a bank account” option is displayed on the home screen. Once clicked, the system displays instructions on how to add a bank account. The customer may enter the account number and link the account, as in 3012, which results in the account getting linked and being available for future transactions, as in 3014.

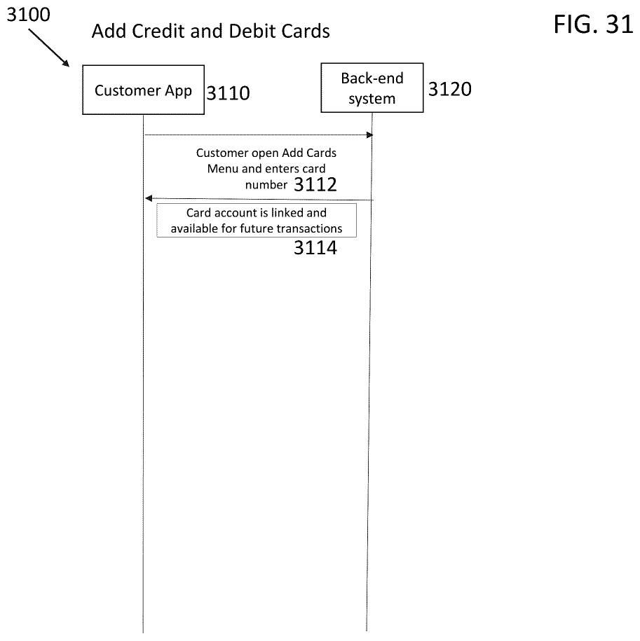

FIG. 31 is an example interactive logic flow 3100 illustrating a method of providing access to add credit and debit cards via the consolidated communication and financial account of FIG. 1, in accordance with an embodiment of this disclosure. Referring to FIG. 31, the interactive logic flow 3100 includes a number of interaction stations, such as a customer application 3110, a back-end system 3120 and the like. The “Add credit or debit cards” process begins when a “Add credit or debit cards” options is displayed on the home screen. Once clicked, the system displays instructions on how to add a credit or a debit card. The customer may enter the card number and link the card, as in 3112, which results in the card getting linked and being available for future transactions, as in 3114.

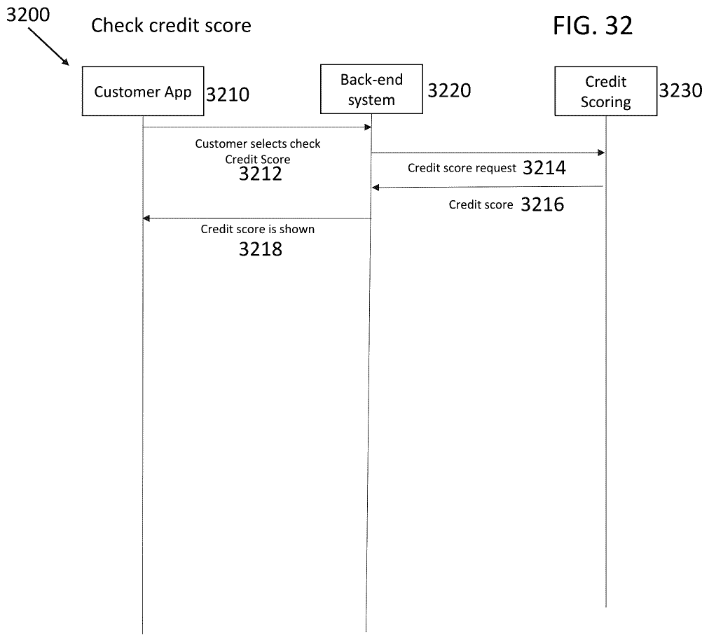

FIG. 32 is an example interactive logic flow 3200 illustrating a method of providing access to credit scores via the consolidated communication and financial account of FIG. 1, in accordance with an embodiment of this disclosure. Referring to FIG. 32, the interactive logic flow 3200 includes a number of interaction stations, such as a customer application 3210, a back-end system 3220, a credit scoring system 3230 and the like. The “Check credit score” process begins when a “Check credit score” option is displayed on the home screen for customer's selection, as in 3212. Once clicked, a request for credit score check may be sent to a credit scoring agency, as in 3214. The credit score may be shared by credit scoring agency, as in 3216 and the score may be shared with the customer, as in 3218.

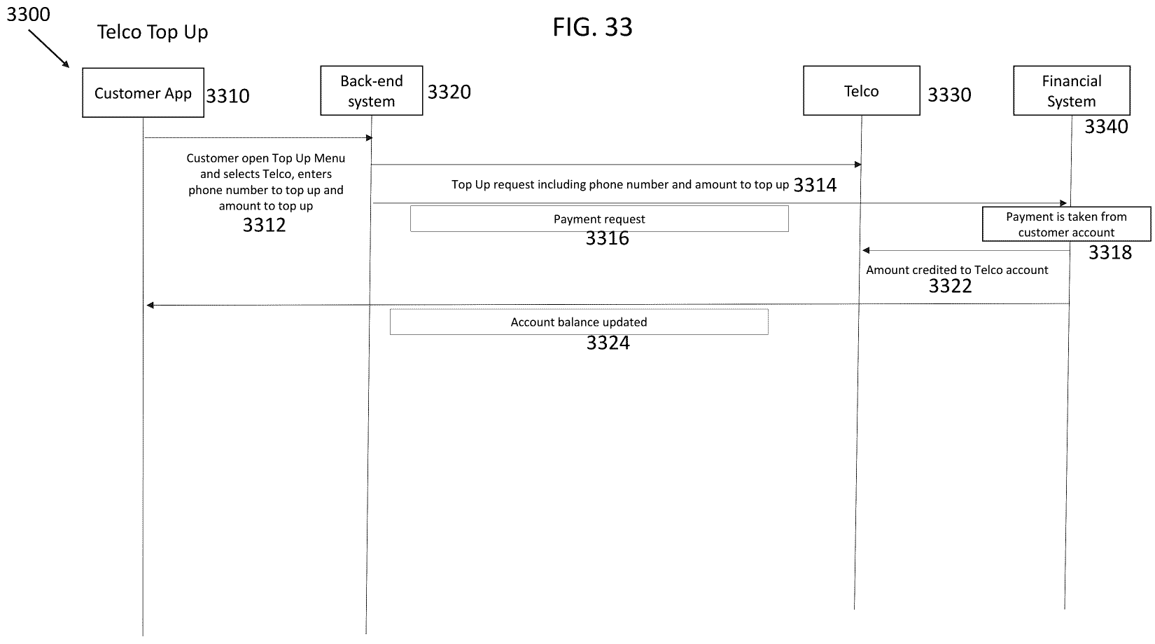

FIG. 33 is an example interactive logic flow 3300 illustrating a method of providing access to telecommunication company (“Telco”) top-up via the consolidated communication and financial account of FIG. 1, in accordance with an embodiment of this disclosure. Referring to FIG. 33, the interactive logic flow 3300 includes a number of interaction stations, such as a customer application 3310, a back-end system 3320, a Telco 3330, a financial system 3340 and the like. The “Telco Top Up” process begins when a “Telco Top Up” option is displayed to a customer on the home screen. Once clicked, the system may display instructions on how to top up telco, as in 3312. The customer may select the option and enter a phone number to top up and an amount to top up, as in 3314. The top up request including the phone number and the amount to top up may be sent to Telco, as in 3316. The payment amount may be automatically withdrawn from the customer's account, as in 3318 and the Telco's account may get credited, as in 3322. The customer's account balance and the Telco's account balance may be updated, as in 3324.

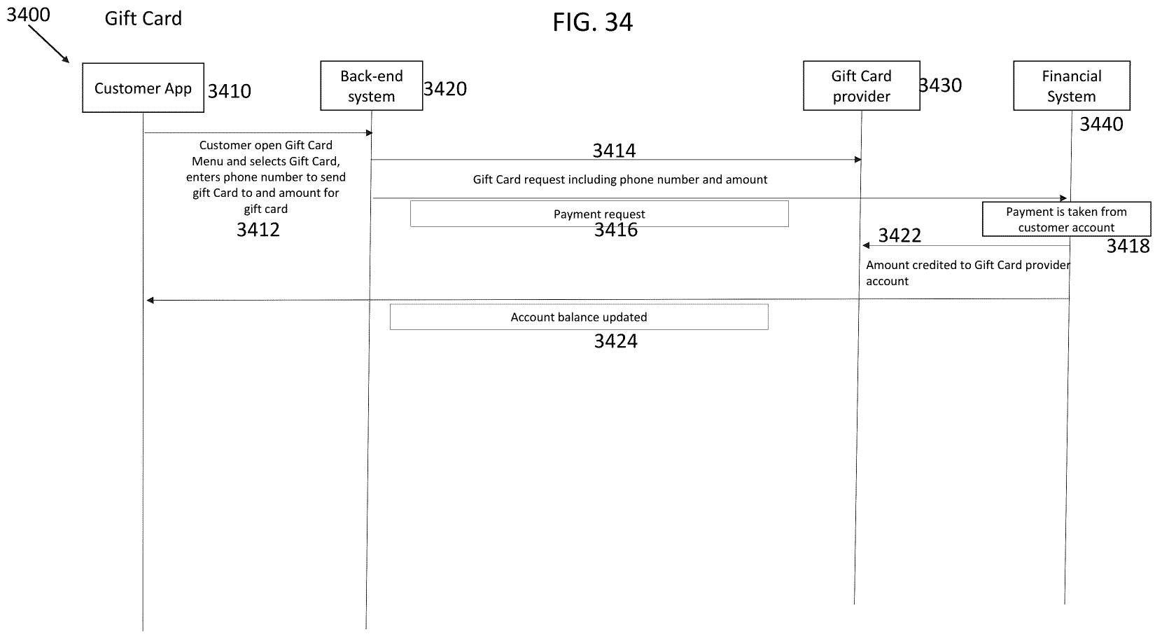

FIG. 34 is an example interactive logic flow 3400 illustrating a method of providing access to gift cards via the consolidated communication and financial account of FIG. 1, in accordance with an embodiment of this disclosure. Referring to FIG. 34, the interactive logic flow 3400 includes a number of interaction stations, such as a customer application 3410, a back-end system 3420, a gift card provider 3430), a financial system 3440 and the like. The “Gift Card Purchase” process begins when a “Gift Card Purchase” option is displayed on the home screen. Once clicked, the system displays a list of all gift cards available. The customer may select a gift card to purchase and the gift card rates may be displayed, as in 3412. The customer may select a specific rate and purchase the gift card. The customer may select a phone number to send the gift card to, as in 3414 and a payment request may be sent, as in 3416. The payment amount may be automatically withdrawn from the customer's account, as in 3418. The purchased gift card may be sent to the receiver and the amount may be credited to the gift card product provider's account, as in 3424. The customer's account balance may be updated, as in 3426.

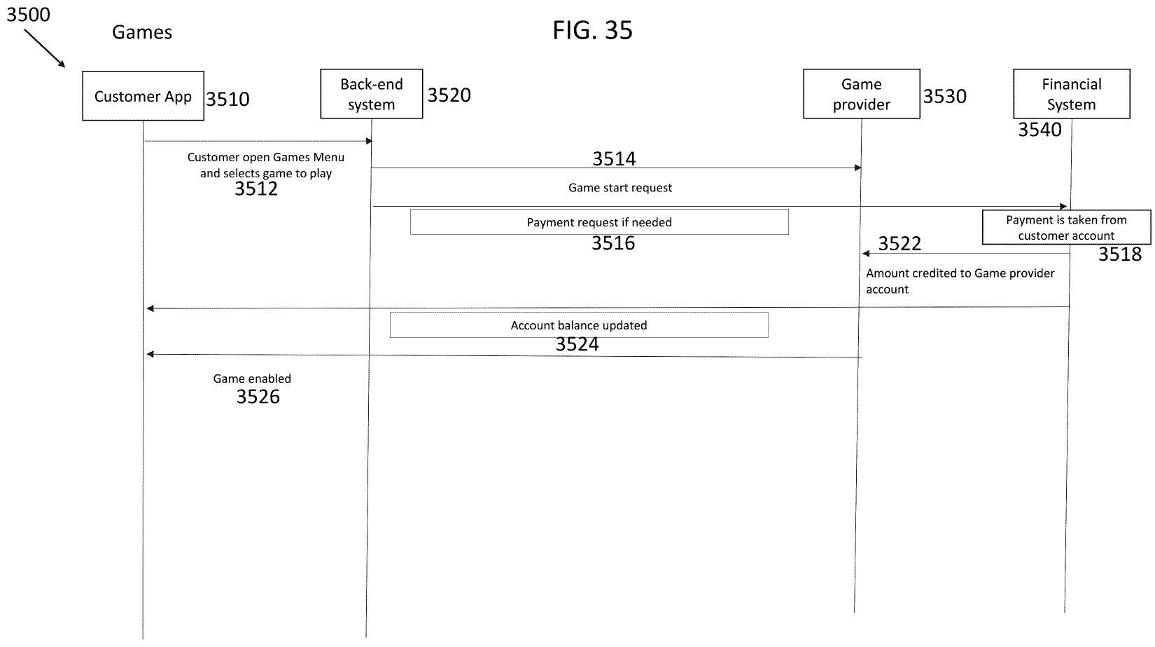

FIG. 35 is an example interactive logic flow 3500 illustrating a method of providing access to games via the consolidated communication and financial account of FIG. 1, in accordance with an embodiment of this disclosure. Referring to FIG. 35, the interactive logic flow 3500 includes a number of interaction stations, such as a customer application 3510, a back-end system 3520, a game provider 3530, a financial system 3540 and the like. The “Games” process begins when a “Games” option is displayed to the user on the home screen. Once clicked, the system displays a list of games available, as in 3512. The customer may select a game to play and a “game start” request is sent to the provider of the game provider, as in 3514. A purchase request may be sent, if needed, as in 3516. The payment amount may be automatically withdrawn from the customer's account, as in 3518 and the amount may be credited to the game provider's account, as in 3522. The customer's account balance may be updated, as in 3524. The game may be enabled and the customer may access the game and begin playing, as in 3526.

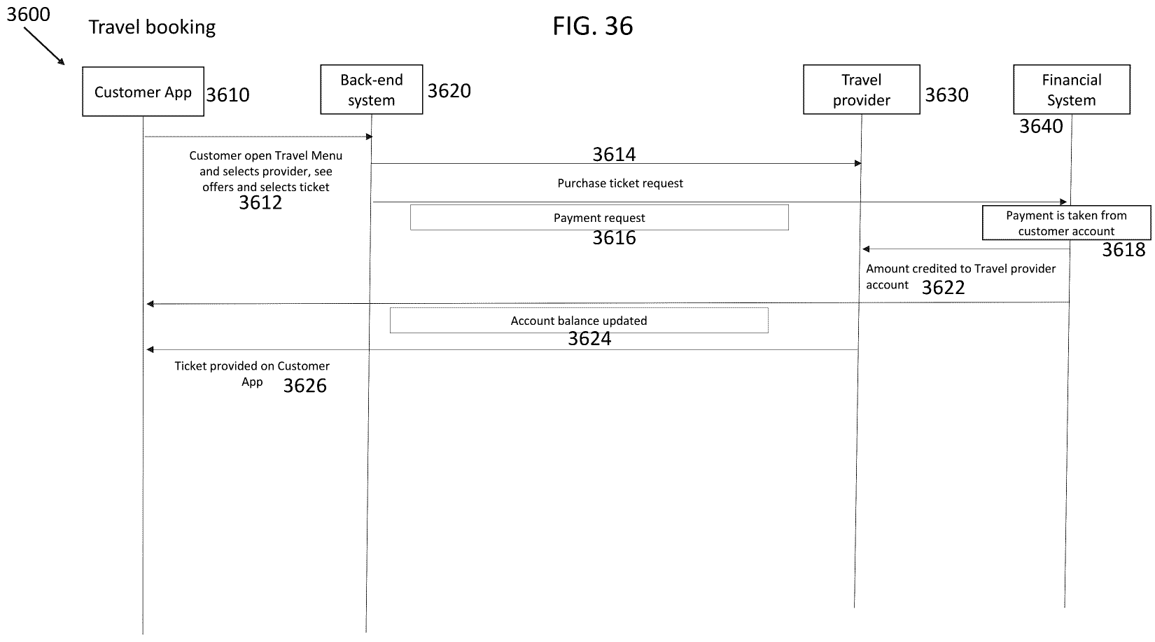

FIG. 36 is an example interactive logic flow 3600 illustrating a method of providing access to travel booking via the consolidated communication and financial account of FIG. 1, in accordance with an embodiment of this disclosure. Referring to FIG. 36, the interactive logic flow 3600 includes a number of interaction stations, such as a customer application 3610, a back-end system 3620, a travel provider 3630, a financial system 3640 and the like. The “Travel” process begins when a “Travel Booking” option is displayed to the user on the home screen. Once clicked, the system displays a list of providers available. The customer may select a provider (for example, bus tickets) and view the offerings, as in 3612. The customer may select a particular ticket, as in 3614 and a “ticket purchase” request is sent to the travel provider, as in 3616. The payment amount may be automatically withdrawn from the customer's account, as in 3618. The amount may be credited to the travel provider's account, as in 3622, the customer's account balance may be updated, as in 3624, and the customer may receive digital tickets on the consolidated communication and financial account, as in 3626. The tickets may be used for traveling.

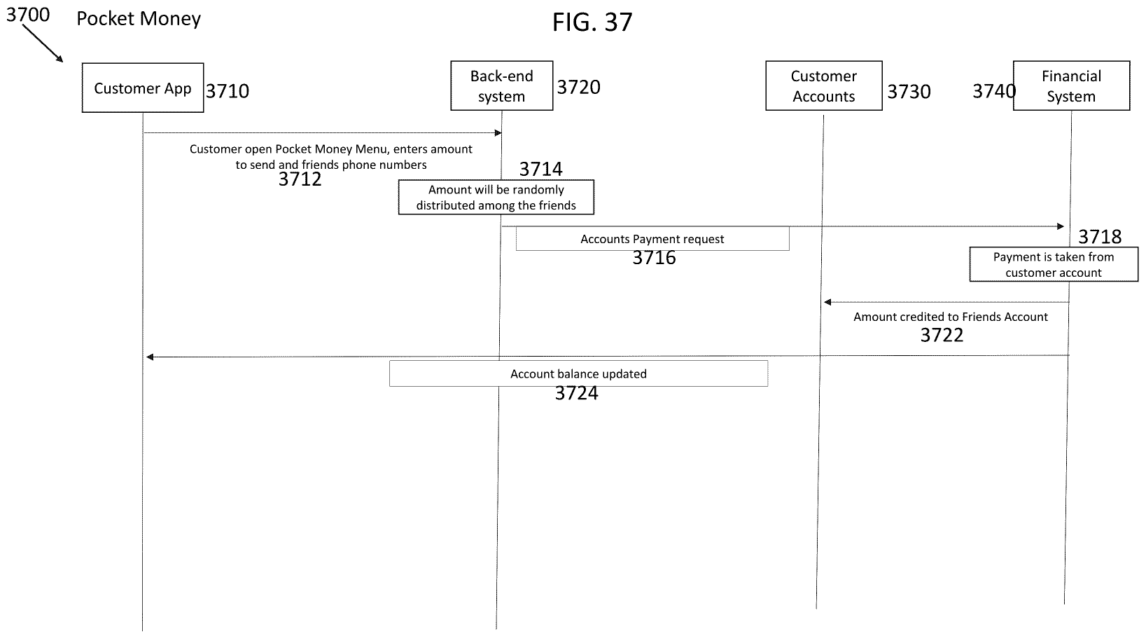

FIG. 37 is an example interactive logic flow 3700 illustrating a method of providing access to pocket money via the consolidated communication and financial account, in accordance with an embodiment of this disclosure. Referring to FIG. 37, the interactive logic flow 3700 includes a number of interaction stations, such as a customer application 3710, a back-end system 3720, customer accounts 3730), a financial system 3740 and the like. The “Pocket Money” process begins when a “Pocket Money” option is displayed to the user on the home screen. Once clicked, the system displays all pocket money options to the customer. The customer may select an amount to send and also a list of receivers to receive the pocket money, as in 3712. The pocket money amount may be randomly distributed among the specified receivers, as in 3714 and sent to their respective accounts registered on the consolidated communication and financial account, as in 3716. The total amount may be withdrawn from the customer's account, as in 3718 and credited to the receivers' individual accounts, as in 3722. The customer's account balance may be updated, as in 3724.

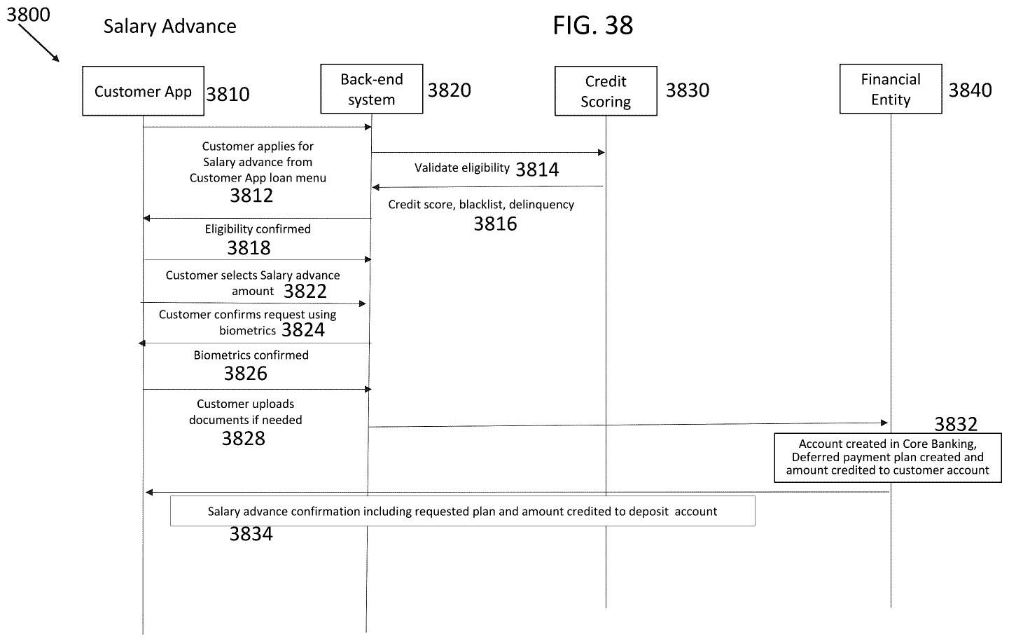

FIG. 38 is an example interactive logic flow 3800 illustrating a method of providing access to salary advance via the consolidated communication and financial account of FIG. 1, in accordance with an embodiment of this disclosure. Referring to FIG. 38, the interactive logic flow 3800 includes a number of interaction stations, such as a customer application 3810, a back-end system 3820, a credit scoring system 3830, a financial entity 3840 and the like. The “Salary Advance” process begins when a “Salary Advance” option is displayed to a customer on the home screen. Once clicked, the system displays all salary advance options available to the customer, as in 3812. The customer may select a specific salary advance option credit scores of the customer may be checked next, as in 3814 and 3816, to appraise the eligibility of the customer, as in 3818, as explained above in relation to the method of FIG. 2.

At this point, the customer may select a salary advance amount, as in 3822. Customer confirms request using instant biometrics, as in 3824. The instant biometrics may be compared with the ones submitted by the customer during onboarding, face or fingerprint depending on device and selection. Once the biometrics are approved, as in 3826, there may be a request for additional documents, as in 3828. Subsequently a loan account may be created in the core banking system, and/or ERP system and/or a CRM system, as in 3832. A deferred payment plan may be created. Further, the customer may be able to view a summary of the loan application that includes the requested loan amount and the approved limit. Once consented, the salary advance amount may be credited to the customer's account, as in 3834.

At this point, the customer may link a payment account and make necessary payments as explained above in relation to the method of FIG. 3.

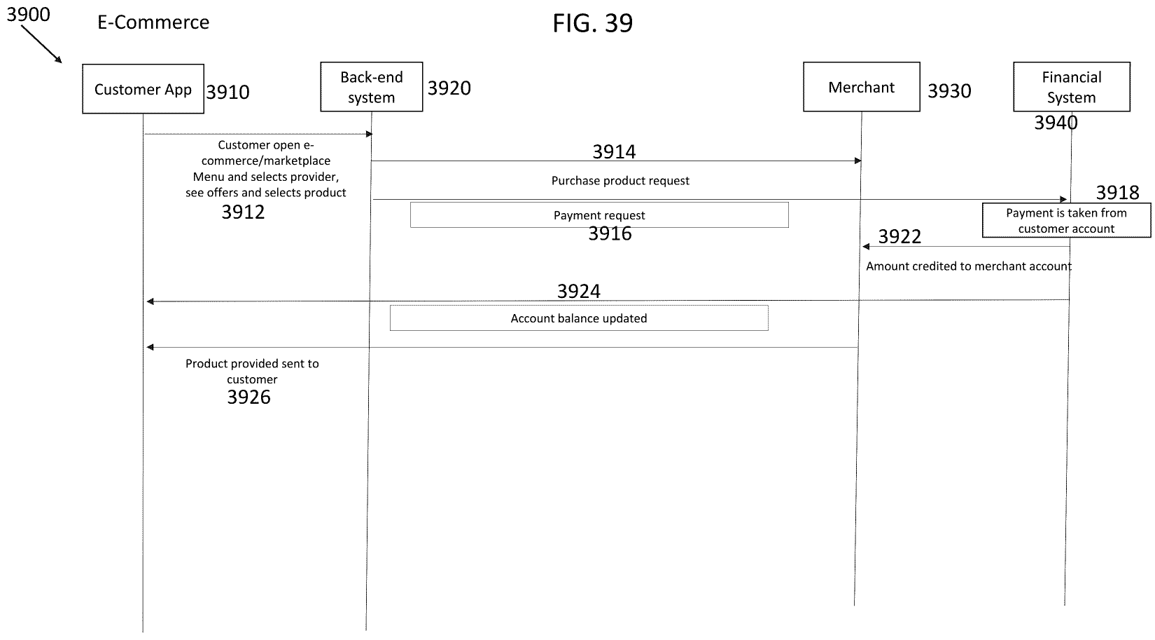

FIG. 39 is an example interactive logic flow 3900 illustrating a method of providing access to e-commerce via the consolidated communication and financial account of FIG. 1, in accordance with an embodiment of this disclosure. Referring to FIG. 39, the interactive logic flow 3900 includes a number of interaction stations, such as a customer application 3910, a back-end system 3920, a merchant 3930, a financial system 3940 and the like. An example “E-Commerce” process begins when an “E-Commerce” option is displayed to a customer on the home screen. Once clicked, the system displays the e-commerce or marketplace menu, as in 3912. The customer may select a provider, view related offers, select a product of interest, as in 3914 and send a payment request, as in 3916. The payment amount may be automatically withdrawn from the customer's account, as in 3918. The amount may be credited to the merchant's account, as in 3922. The customer's account balance may be updated, as in 3924 and the customer may receive the product at a selected or specified location, as in 3926.

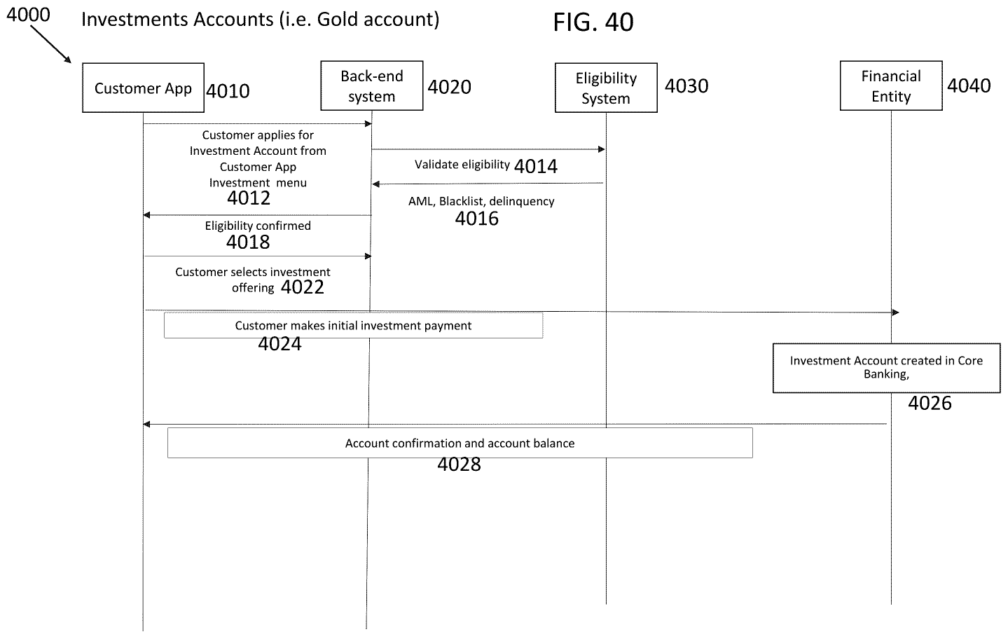

FIG. 40 is an example interactive logic flow 4000 illustrating a method of providing access to investments accounts via the consolidated communication and financial account of FIG. 1, in accordance with an embodiment of this disclosure. Referring to FIG. 40, the interactive logic flow 4000 includes a number of interaction stations, such as a customer application 4010, a back-end system 4020, an eligibility system 4030, a financial entity 4040 and the like. An example “Investment Account” process begins when an “Apply for Investment Account” option menu is displayed to a customer on the home screen. Once clicked, the system displays all different offerings available to the customer (such as education, healthcare and the like). The customer selects a type of investment account, as in 4012. Credit scores of the customer may be checked next to appraise the eligibility of the customer, as in 4014 and 4016, and as explained above in relation to the method of FIG. 2. After eligibility is confirmed, as in 4018, the customer may see a summary of all terms and conditions related to an investment account, as in 4022.

At this point, the customer may subscribe and make the initial investment, as in 4024. An investment account may be created in a core banking system and/or an ERP system and/or a CRM system, as in 4026. A confirmation message may be shared with the customer in the consolidated communication and financial account. The investment account balance may be part of the customer products on the home page. Further, there may be a tracker that notifies the customer with the balance remaining to achieve the investment goal, as in 4028. Responsive to successful receipt of a minimum number of deposits, the system may automatically offer a subsequent (second or third and so forth) investment account to the customer. The terms of the subsequent investment may be configured based on a history of the customer's account usage and deposits.

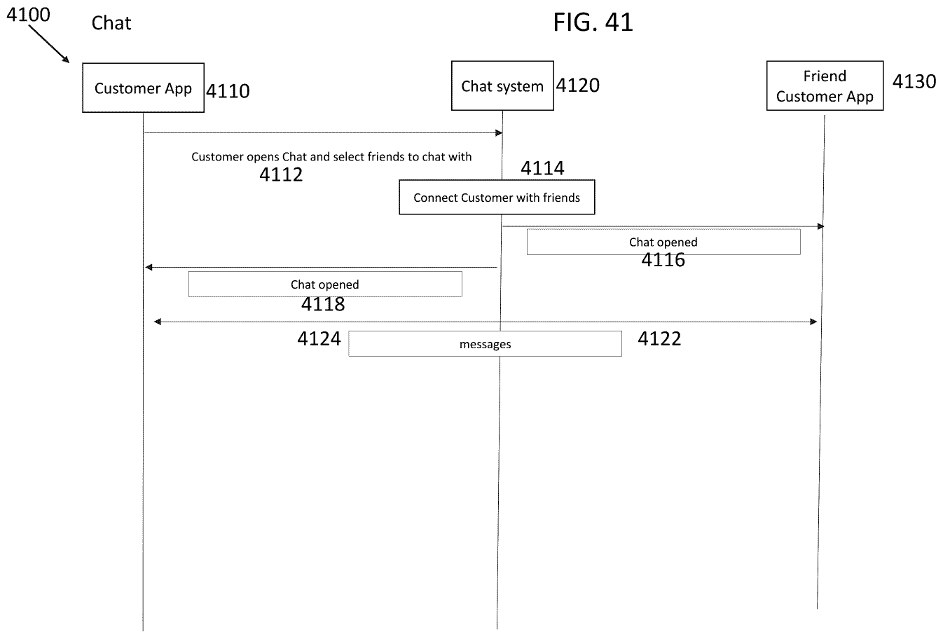

FIG. 41 is an example interactive logic flow 4100 illustrating a method of providing access to chats via the consolidated communication and financial account of FIG. 1, in accordance with an embodiment of this disclosure. Referring to FIG. 41, the interactive logic flow 4100 includes a number of interaction stations, such as a customer application 4110, a back-end system 4120, a friend customer application system 4130 and the like. An example “Chat” process begins when a “Chat” option is displayed to a customer on the home screen. Once clicked, the system displays a chat screen to the customer. The customer may open the chat and select individuals to chat with, as in 4112. The chat system may connect the customer with the select individuals, as in 4114. In operation, the chat window may be opened on the application pages 4116, 4118, and so on of the select individuals and on the customer's application and messages 4122, 4124, and so on may be exchanged.

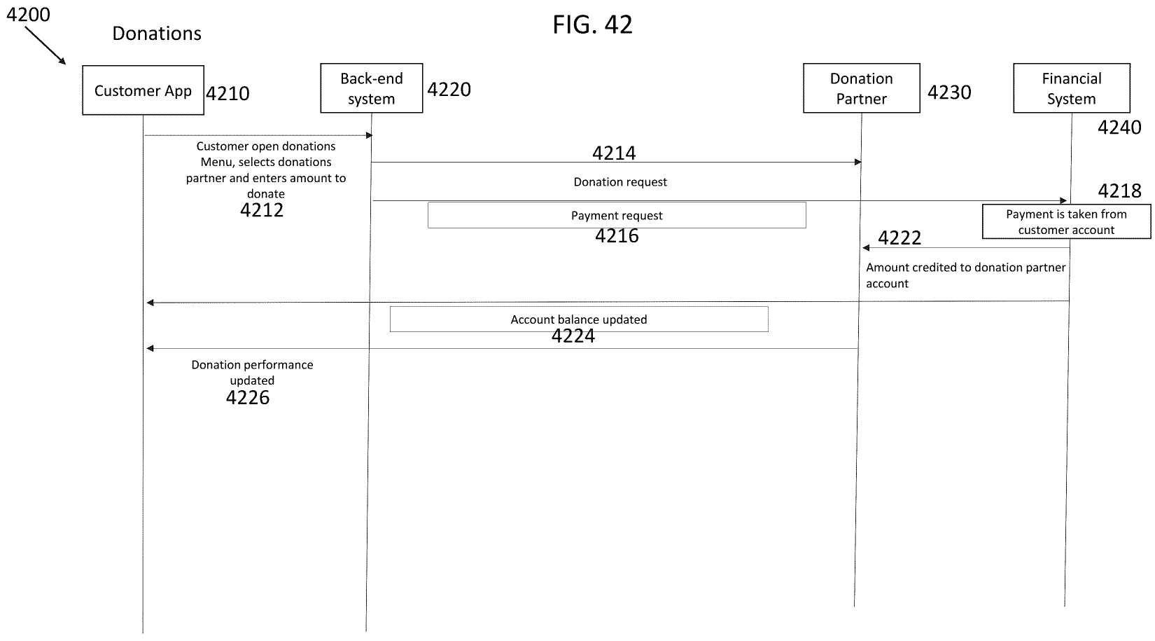

FIG. 42 is an example interactive logic flow 4200 illustrating a method of providing access to donations via the consolidated communication and financial account of FIG. 1, in accordance with an embodiment of this disclosure. Referring to FIG. 42, the interactive logic flow 4200 includes a number of interaction stations, such as a customer application 4210, a back-end system 4210), a donation partner 4230, a financial system 4240 and the like. An example “Donation” process begins when a “Donations” option is displayed to a customer on the home screen. Once clicked, the donations pages are displayed to the customer. The customer may select a donation partner and enter an amount to donate, as in 4212. A donation request 4214 may be sent to the donation partner 4230 and simultaneously, a donation request 4216 may be sent to the financial system 4240. The payment amount may be automatically withdrawn from the customer's account, as in 4218 and the amount may be credited to the donation partner's account, as in 4222. The customer may track the performance of the donation partner and the balance remaining, to achieve a donation goal. Specifically, the customer's account balance may be updated, as in 4224 and a donation goal performance tracker may be updated, as in 4226.

The present disclosure may be implemented as a system, a method, and/or a computer program product operationalized on a communication device or a mobile telephone. The computer program product may include a computer readable storage medium (or media) such as a memory having computer readable program instructions stored thereon for causing a processor to carry out certain aspects of the present disclosure.

The computer readable storage medium such as the memory can be a tangible device that can retain and store instructions for use by an instruction execution device (such as the data processor(s) of the computer, as shown as the device of FIG. 1. The computer readable storage medium may be on a same or different chip as the data processor, and for example may be but is not limited to an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In the above description, numerous specific details such as resource partitioning/sharing/duplication implementations, types and interrelationships of system components, and logic partitioning/integration choices are set forth in order to provide a more thorough understanding. Embodiments disclosed herein may be practiced without such specific details, however. In other instances, control structures, logic implementations, opcodes, means to specify operands, and full software instruction sequences have not been shown in detail since those of ordinary skill in the art, with the included descriptions, may be able to implement what is described without undue experimentation.

References in the specification to “one implementation,” “an implementation,” “an example implementation,” etc., indicate that the implementation described may include a particular feature, structure, or characteristic, but every implementation may not necessarily include the particular feature, structure, or characteristic. Moreover, such phrases are not necessarily referring to the same implementation. Further, when a particular feature, structure, and/or characteristic is described in connection with an implementation, one skilled in the art would know to affect such feature, structure, and/or characteristic in connection with other implementations whether or not explicitly described.

For example, the figure(s) illustrating flow diagrams sometimes refer to the figure(s) illustrating block diagrams, and vice versa. Whether or not explicitly described, the alternative implementations discussed with reference to the figure(s) illustrating block diagrams also apply to the implementations discussed with reference to the figure(s) illustrating flow diagrams, and vice versa. At the same time, the scope of this description includes implementations, other than those discussed with reference to the block diagrams, for performing the flow diagrams, and vice versa.

The detailed description and claims may use the term “coupled.” along with its derivatives. “Coupled” is used to indicate that two or more elements, which may or may not be in direct physical or electrical contact with each other, co-operate or interact with each other.

While the flow diagrams in the figures show a particular order of operations performed by certain implementations, such order is illustrative and not limiting (e.g., alternative implementations may perform the operations in a different order, combine certain operations, perform certain operations in parallel, overlap performance of certain operations such that they are partially in parallel, etc.).

While the above description includes several example implementations, the invention is not limited to the implementations described and can be practiced with modification and alteration within the spirit and scope of the appended claims. The description is thus illustrative instead of limiting.

What is claimed is:

1. A method of providing access to a multi-platform consolidated communication and financial account, the method comprising:
receiving identifying information from a user;
performing a first verification of the user with a first service provider using the identifying information to verify an identity of the user;
performing a second verification of the identity of the user via a second service provider, the second service provider being separate from the first service provider;
responsive to a successful first verification and a successful second verification of the identity of the user, creating a multi-platform consolidated communication and financial account;
providing a communication device to the user, the communication device providing access to one or more communication networks;
creating a record in a user device management system, the record specifying the identity of the user, the account, and a unique identifier of the communication device provided to the user; and
linking a payment account of the user to the device to allow for payment by the user via the device.

2. The method of claim 1 further comprising preventing opening a second account by the same user and responsive to an attempt by the user to create the second account, generating an error.

3. The method of claim 1 further comprising, prior to performing the first verification of the user, receiving a request from the user to identify an authorized agent to establish an account for the user on the device.

4. The method of claim 1, wherein the step of performing the first verification of the user further comprises;
receiving confirmation of the identifying information from an agent in physical proximity to the user, wherein the confirmation indicates that the agent has independently verified the accuracy of the identifying information;
sending the identifying information to a telephone service provider;
receiving, from the telephone service provider, a confirmation that the identifying information matches a user record of an account at the telephone service provider.

5. The method of claim 1, further comprising:
receiving a request for a deferred payment plan from the device for purchase of an item from a merchant;
responsive to the request, confirming an eligibility of the user;
responsive to confirming that the user is eligible, verifying a biometric signature of the user via a third service provider separate from the first service provider and the second service provider;
responsive to successful verification of the biometric signature of the user, providing payment for the item to the merchant and adding the deferred payment plan to an account of the user associated with the device.

6. The method of claim 1, further comprising providing access to device financing via self-onboarding on the multi-platform consolidated communication and financial account, the providing access to device financing comprising:
the user signing up on the user device management system;
the user providing identifying information to the user device management system;
appraising a credit eligibility of the user via a third service provider separate from the first service provider and the second service provider;
responsive to user being eligible for credit, forwarding the user to a registering agent;
the user completing onboarding with assistance from the registering agent;
the user paying a dollar amount from a user account as cost of a membership via the user device management system; and
updating the user account.

7. The method of claim 1, further comprising providing access to device financing via an agent-assisted onboarding on the multi-platform consolidated communication and financial account, the providing access to device financing comprising:
the user signing up on the user device management system;
the user providing identifying information to the user device management system;
a registering agent receiving the identifying information;
the registering agent validating the user's identity;
the registering agent appraising a credit eligibility of the user via a third service provider separate from the first service provider and the second service provider;
responsive to the user being eligible for credit, completing onboarding of the user;
the user paying a dollar amount from a user account as cost of a membership with consumer based financial application; and
updating the user account.

8. The method of claim 1, further comprising providing access to a loan via the multi-platform consolidated communication and financial account, the providing access to a loan comprising:
displaying a plurality of financial offerings available to the user;
the user selecting a loan option from the plurality of financial offerings;
appraising a credit eligibility of the user via a third service provider separate from the first service provider and the second service provider;
responsive to user being eligible for credit, forwarding the user to a loan agent;
the user completing a loan registration with assistance from the loan agent;
the user paying a dollar amount from a user account as cost of the loan;
updating the user account;
monitoring for timely payments to the loan; and
automatically offering additional loan based on the payments to the loan.

9. The method of claim 8, wherein the appraising a credit eligibility of the user comprises obtaining a credit score of the user, the credit score of the user being above a minimum threshold, initiating the loan to the user's authenticated identity.

10. The method of claim 9, wherein the loan comprises at least one of:
a short-term cash loan,
an asset financing loan,
a BNPL loan,
a bill payment financing,
a business loan, and
a medium-term and long-term cash loan.

11. The method of claim 10, further comprising:
responsive to determining that the loan is in default, blocking access to the one or more communication networks by the device.

12. The method of claim 10, further comprising:
responsive to successful use of the device and receipt of a minimum number of timely loan payments, offering a second loan to the user, wherein terms of the second loan are based on a user history of usage of the device.

13. The method of claim 1, further comprising providing access to insurance via the multi-platform consolidated communication and financial account, the providing access to insurance comprising:
displaying a plurality of financial offerings available to the user;
the user selecting an insurance option from the plurality of financial offerings;
appraising an insurance eligibility of the user via a third service provider separate from the first service provider and the second service provider;
responsive to user being eligible for insurance, forwarding the user to an insurance agent;
the user completing an insurance registration with assistance from the insurance agent;
the user paying a dollar amount from a user account as cost of the insurance;
updating the user account;
monitoring for timely payments to the insurance; and
automatically offering additional insurance based on the payments to the insurance.

14. The method of claim 1, further comprising providing access to goal-based savings via the multi-platform consolidated communication and financial account, the providing access to goal-based savings comprising:
displaying a plurality of financial offerings available to the user;
the user selecting a goal-based savings option from the plurality of financial offerings;
appraising a credit eligibility of the user via a third service provider separate from the first service provider and the second service provider;
responsive to user being eligible for credit, forwarding the user to a goal-based savings registering agent;
the user completing a goal-based saving scheme registration with assistance from the goal-based savings registering agent;
the user paying a dollar amount from a user account as cost of the goal-based saving scheme;
updating the user account;
monitoring for timely payments to the goal-based saving scheme; and
automatically offering additional goals based on the payments to the goal-based saving scheme.

15. The method of claim 1, further comprising providing access to a consumer card via the multi-platform consolidated communication and financial account, the providing access to a consumer card comprising:
displaying a plurality of financial offerings available to the user;
the user selecting a consumer card option from the plurality of financial offerings;
appraising a credit eligibility of the user via a third service provider separate from the first service provider and the second service provider;
responsive to user being eligible for credit, forwarding the user to a consumer card registering agent; and
the user completing consumer card registration with assistance from the consumer card registering agent;
the user paying a dollar amount from a user account as cost of the consumer card service;
updating the user account;
monitoring for timely payments to the consumer card; and
automatically offering additional loans based on the payments to the consumer card.

16. The method of claim 15, wherein the consumer card comprises a credit card or a debit card.

17. The method of claim 1, further comprising providing access to a telehealth service via the multi-platform consolidated communication and financial account, the providing access to a telehealth service comprising:
displaying a plurality of healthcare offerings available to the user;
the user selecting a telehealth option from the plurality of healthcare offerings and selecting a doctor;
checking an availability of the telehealth service using a telehealth doctor's calendar, the telehealth service offered by a third service provider separate from the first service provider and the second service provider;
reserving an appointment on the telehealth doctor's calendar and confirming the appointment with the user;
scheduling the appointment on the doctor's calendar and on the user's calendar;
the user paying a dollar amount from a user account as cost of the telehealth service; and
updating the user account.

18. The method of claim 1, further comprising providing access to a doctor's appointments via the multi-platform consolidated communication and financial account, the providing access to a doctor's appointments comprising:
displaying a plurality of healthcare offerings available to the user;
the user selecting a doctor's appointments option from the plurality of healthcare offerings and selecting a doctor;
checking an availability of doctor's from the doctor's calendar via a third service provider separate from the first service provider and the second service provider;
reserving an appointment on the doctor's calendar and confirming the appointment with the user;
scheduling the appointment on the doctor's calendar and on the user's calendar;
the user paying a dollar amount from a user account as cost of the telehealth service; and
updating the user account.

19. The method of claim 1, further comprising providing access to a pharmacy via the multi-platform consolidated communication and financial account, the providing access to a pharmacy comprising:
displaying a plurality of healthcare offerings available to the user;
the user selecting a pharmacy from the plurality of healthcare offerings, the pharmacy operated by a third service provider separate from the first service provider and the second service provider;
the user selecting a product from the pharmacy for purchase;
the user paying a dollar amount from a user account as price of the product;
updating the user account; and
shipping the product to the user.

20. The method of claim 1, further comprising providing access to a merchant payment via the multi-platform consolidated communication and financial account, the providing access to a merchant payment comprising:
displaying a plurality of options to the user to make the merchant payment;
displaying instructions on how to make the merchant payment via a third service provider separate from the first service provider and the second service provider;
the user scanning a QR code related to the merchant payment;
the user paying the dollar amount from a user account to make the merchant payment; and
updating the user account.

21. The method of claim 1, further comprising providing access to a bill payment via the multi-platform consolidated communication and financial account, the providing access to a bill payment comprising:
displaying a plurality of options to the user to make the bill payment;
displaying instructions on how to make the bill payment via a third service provider separate from the first service provider and the second service provider;
the user selecting one of the plurality of options to make the bill payment;
the user entering a bill payment account number and a dollar amount to pay;
the user paying the dollar amount from a user account; and
updating the user account.

22. The method of claim 1, further comprising providing access to government payments via the multi-platform consolidated communication and financial account, the providing access to government payments comprising:
displaying a plurality of options to the user to make government payments;
displaying instructions on how to make government payments via a third service provider separate from the first service provider and the second service provider;
the user selecting one of the plurality of options to make the government payment;
the user entering a user account number to make the payment from and a dollar amount to pay;
the user paying the dollar amount from the user account;
updating the user account; and
the user optionally subscribing for an automated payment on a recurring due date.

23. The method of claim 1, further comprising providing access to add a bank account via the multi-platform consolidated communication and financial account, the providing access to add a bank account comprising:
displaying a plurality of options to the user to add a bank account;
displaying instructions on how to add the bank account via a third service provider separate from the first service provider and the second service provider;
the user selecting one of the plurality of options to add the bank account;
the user entering an associated number of the bank account; and
linking the bank account with a purchase account of the user; and
making the bank account available for future transactions of the user.

24. The method of claim 1, further comprising adding a consumer card via the multi-platform consolidated communication and financial account, the adding a consumer card comprising:
displaying a plurality of options to the user to add a consumer card;
displaying instructions on how to add a consumer card via a third service provider separate from the first service provider and the second service provider;
the user selecting one of the plurality of options to add the consumer card;
the user entering an associated number of the consumer card; and
linking the consumer card with a purchase account of the user; and
making the consumer card available for future transactions of the user.

25. The method of claim 24, wherein the consumer card comprises a credit card or a debit card.

26. The method of claim 1, further comprising providing access to credit score via the multi-platform consolidated communication and financial account, the providing access to credit score comprising:
displaying a plurality of options to the user to check a credit score of the user via a third service provider separate from the first service provider and the second service provider;
the user selecting one of the plurality of options to check the credit score;
sending a credit score check request to a credit score system;
receiving the credit score of the user from the credit score system; and
displaying the credit score of the user.
